(19) United States
(12) Patent Application Publication
(10) Pub. No.: US 2021/0278405 A1
(43) Pub. Date: Sep. 9, 2021

(54) IDENTIFICATION OF METABOLOMIC SIGNATURES IN URINE SAMPLES FOR TUBERCULOSIS DIAGNOSIS

(71) Applicants: CONSORCIO CENTRO DE INVESTIGACIÓN BIOMÉDICA EN RED, M.P., Madrid (ES); FUNDACIÓ INSTITUT D'INVESTIGACIÓ EN CIÈNCIES DE LA SALUT GERMANS TRIAS I PUJOL, Badalona (ES)

(72) Inventors: José Luís IZQUIERDO GARCÍA, Madrid (ES); José DOMÍNGUEZ, Badalona (ES); Cristina PRAT AYMERICH, Badalona (ES); Patricia COMELLA DEL BARRIO, Badalona (ES)

(21) Appl. No.: 17/253,582

(22) PCT Filed: Jun. 18, 2019
(86) PCT No.: PCT/EP2019/066055

§ 371 Date: Dec. 17, 2020

(30) Foreign Application Priority Data
Jun. 18, 2018 (EP) 18382437.4

(51) Int. Cl. G01N 33/569; G01N 24/08; G01R 33/465

(52) U.S. Cl. CPC G01N 33/5695; G01R 33/465; G01N 24/08

(57) ABSTRACT

The authors of the present invention have identified a series of metabolic markers present in the urine samples collected from patients diagnosed of tuberculosis (TB, n=19), respiratory infections caused by Streptococcus pneumoniae (R1, n=25) and healthy controls (HC, n=29). These metabolic markers selected are significantly differentiated between Healthy Controls (HC) and patients diagnosed of tuberculosis, between tuberculosis patients versus patients affected by respiratory infections caused by S. pneumoniae, and between patients affected by respiratory infections caused by S. pneumoniae and HC. These metabolic markers can thus be used in a non-invasive diagnostic method identifying and classifying patients.

### FIELD OF THE INVENTION

The present invention relates to the field of diagnostics and, more in particular the non-invasive diagnosis of tuberculosis patients. In particular, the present invention relates to the use of urine metabolomics biomarkers to differentiate tuberculosis patients from Healthy Controls (HC) or from patients suffering from respiratory infections caused by Streptococcus pneumoniae.

### BACKGROUND OF THE INVENTION

The invention relates to a method of diagnosis of mycobacterial infection, particularly Mycobacterium tuberculosis infection. It also relates to a kit which can be used to carry out the diagnostic method.

Current diagnostic tests for tuberculosis disease are either slow or unreliable. Tests that rely on the identification of the M. tuberculosis which causes tuberculosis are slow because culturing of the M. tuberculosis can take up to 8 weeks. In some cases it proves impossible to culture the bacteria. In addition the obtaining of samples to detect the presence of the M. tuberculosis often requires invasive procedures. The detection of DNA from the bacteria by molecular methods is also an alternative, but it requires specific equipment, trained technicians and it is not available in all laboratories.

An alternative test is the tuberculin skin test (TST) or Mantoux test which is based on the detection of delayed type hypersensitivity (DTH) response to an intradermal administration of a Purified Protein Derivative of the M. tuberculosis. Although this test takes less time than tests which rely on identification of the M. tuberculosis, it is less reliable because of the widespread use of BCG as a vaccine against tuberculosis. BCG is closely related to M. tuberculosis and therefore individuals who have been vaccinated with BCG can react positively to a TST. In addition a large proportion of people with active tuberculosis are not detected by a TST because of cutaneous immune anergy. Thus TST has a low specificity and sensitivity. In addition, the TST don't distinguish between latent tuberculosis and active tuberculosis.

Other known methods to carry out the diagnostic method comprise incubating a blood sample from an animal with mycobacterial antigens, and detecting the presence of cell-mediated immune-response resulting from the incubation or detecting antibodies to mycobacterial antigens, respectively. It has turned out, however that different antigens detect a partially differing population of tuberculosis infected animals. The sensitivity of these antigens appears lower than the sensitivity of tuberculin. As a consequence in some situations, assays using the antigens can produce false negative results and do not distinguish between latent tuberculosis and active tuberculosis.

In view of the above it is an object of the invention to provide improved diagnostic methods which allows the design of methods for the diagnosis of tuberculosis with increased specificity and sensitivity in non-invasive biological samples.

### BRIEF DESCRIPTION OF THE INVENTION

The authors of the present invention have identified a series of metabolic markers present in the urine samples collected from patients diagnosed of tuberculosis (TB, n=19), respiratory infections caused by S. pneumoniae (RI, n=25) and healthy controls (HC, n=29). These metabolic markers selected are significantly differentiated between Healthy Controls (HC) and patients diagnosed of tuberculosis, between tuberculosis patients versus patients affected by respiratory infections caused by S. pneumoniae, between patients affected by respiratory infections caused by S. pneumoniae and HC and can distinguish between latent tuberculosis and active tuberculosis. These metabolic markers can thus be used in a non-invasive diagnostic method identifying and classifying patients.

### BRIEF DESCRIPTION OF THE FIGURES

FIG. 1. PCA scoring performed on 1H High Field NMR spectra of urine samples from Tuberculosis, Healthy Control and Respiratory Infection subjects are clearly separated in three clusters.

FIG. 2. Top: PCA score plot (Left) and PCA loadings correlation plot (right) performed on 1H High Field NMR spectra of urine samples from Tuberculosis, Healthy Control subjects. NMR regions with absolute value of the loading higher than 0.14 were selected as potential biomarkers (Hotteling's T2 tests, p<0.01). Bottom: PCA score plot (Left) and PCA loadings correlation plot (right) performed on 1H High Field NMR spectra of urine samples from Tuberculosis, Respiratory Infections subjects. NMR regions with absolute value of the loading higher than 0.11 were selected as potential biomarkers (Hotteling's T2 tests, p<0.01).

FIG. 3. A: Representative 1D-1H NMR spectrum of urine samples from tuberculosis subjects acquired by a high field (Top) and a low field (Bottom) NMR spectrometers. The figure highlights the metabolites selected to build the predictive models. B: Representative 2D-1H-13C HSQC NMR spectrum of urine samples from tuberculosis subjects acquired by a high field NMR spectrometers.

FIG. 4. PLS-DAs were developed as diagnostic models of tuberculosis based on the intensity of selected High field NMR regions. A) PLS-DA of Tuberculosis (TB) vs Healthy Control (Ctrl) subjects. B) PLS-DA of Tuberculosis (TB) vs Respiratory Infection (RES) subjects. In black the samples used to train the model. In blue the samples used to validate the model.

FIG. 5. PLS-DAs were developed as diagnostic models of tuberculosis based on the intensity of selected metabolites quantified by High Field NMR. A) PLS-DA of Tuberculosis (TB) vs Healthy Control (Ctrl) subjects. B) PLS-DA of Tuberculosis (TB) vs Respiratory Infection (RES) subjects. In black the samples used to train the model. In blue the samples used to validate the model.

FIG. 6. PLS-DA was developed as diagnostic model of Tuberculosis (TB) vs Healthy Control (Ctrl) subjects based on the intensity of selected low field NMR regions. In black the samples used to train the model. In blue the samples used to validate the model.

FIG. 7. Results in Children: TB vs Healthy controls. PLS-DA was developed as diagnostic model of Tuberculosis (TB) vs Healthy Control (Ctrl) subjects based on the intensity of selected high field NMR regions in pediatric population

FIG. 8. Results in Children: TB vs Latent TB Infection. PLS-DA was developed as diagnostic model of Tuberculosis (TB) vs latent TB infected (LTBI) subjects based on the intensity of selected high field NMR regions in pediatric population

FIG. 9. Results in Children: TB vs Latent TB Infection vs Healthy controls. PLS-DA was developed as diagnostic model of Tuberculosis (TB) vs latent TB infected (LTBI) subjects based on the intensity of selected high field NMR regions in pediatric population

FIG. 10. Results in adults: TB vs Latent TB Infection. PLS-DA was developed as diagnostic model of Tuberculosis vs latent TB infected (Latent) vs Healthy Control (non-latently TB infected) subjects based on the intensity of selected high field NMR regions in pediatric population

FIG. 11. PLS-DA was developed as diagnostic model of Tuberculosis vs latent TB infected (infection) vs non-latent TB infected (Non-infection) subjects based on the intensity of selected low field NMR regions in adults. In black the samples used to train the model. In red the samples used to validate the model.

FIG. 12. PLS-DA was developed to evidence the difference between Tuberculosis, healthy controls (CTRL) and patients with active TB patients with more than 40 days of TB treatment, based on the intensity of selected low field NMR regions in adults.

FIG. 13. Classification scheme.

### DESCRIPTION OF THE INVENTION

### Diagnostic Methods of the Invention

Before the present methods are described, it is to be understood that this invention is not limited to particular methods, and experimental conditions described, methods and conditions may vary. It is also to be understood that the terminology used herein is for purposes of describing particular embodiments only, and is not intended to be limiting, since the scope of the present invention will be limited only in the appended claims.

The authors of the present invention have identified a series of metabolic markers present in the urine samples collected from patients diagnosed of tuberculosis (TB, n=19), respiratory infections caused by S. pneumoniae (RI, n=25) and healthy controls (HC, n=29). These metabolic markers selected are significantly differentiated between Healthy Controls (HC) and patients diagnosed of tuberculosis, between tuberculosis patients versus patients affected by respiratory infections caused by S. pneumoniae, and between patients affected by respiratory infections caused by S. pneumoniae and HC. These metabolic markers can thus be used in a non-invasive diagnostic method for identifying and classifying patients. In particular, the invention relates to a diagnostic method to distinguish between: tuberculosis patients versus HC, between tuberculosis patients versus patients affected by respiratory infections caused by S. pneumoniae, between patients affected by respiratory infections caused by S. pneumoniae and HC and can further distinguish between latent tuberculosis and active tuberculosis and between latent tuberculosis and healthy controls, based in three different urine biomarkers profiles. Each of these biomarker profiles is identified and explained below.
Urine Biomarker Profile for Identifying and Classifying Tuberculosis Patients Versus HC

The authors of the present invention have determined that by using the different subsets of metabolites identified below, and preferably applying PLS-DA, the following discrimination results were obtained for tuberculosis patients versus HC:
Citrate and creatinine: TB predictive value: 71.43 SD=13.85 Sensitivity=71.43%(10.40) Specificity=75.00% (6.06);
Citrate, creatinine and hippurate: TB predictive value: 79.57 SD=11.88 Sensitivity=80.17%(9.40) Specificity=90.08% (5.44).
Citrate, creatinine, mannitol and hippurate: TB predictive value: 86.14 SD=9.88 Sensitivity=93.44%(7.33) Specificity=93.50% (4.45).
Citrate, creatinine, mannitol, hippurate and glucose: TB predictive value: 86.29 SD=9.06 Sensitivity=93.44%(7.19) Specificity=93.55% (4.10).
Citrate, creatinine, mannitol, hippurate, glucose, Phenylalanine, Creatine and 2-Aminoadipic Acid. TB predictive value: 79.29% SD(3.58%); Sensitivity: 91.22% SD(7.99%); Specifity:90.52% SD(5.05%).

Therefore, a first aspect the invention relates to an in vitro method to classify a subject in need thereof, between patients suffering from tuberculosis, that is to say infected with M: tuberculosis and preferably suffering the symptomatology of the disease, vs HC (healthy controls and/or subjects not infected with M. tuberculosis and/or not suffering the symptomatology of the disease, in this specific context of the invention HC includes latent tuberculosis) (from hereinafter “first classification method of the invention”), that comprises the in vitro determination of the levels of at least citrate and creatinine in a urine sample taken from the subject. Preferably, the in vitro classification method is based on the in vitro determination of the levels of at least citrate, creatinine and hippurate in a urine sample taken from the subject. More preferably, the in vitro classification method is based on the in vitro determination of the levels of at least citrate, creatinine, mannitol and hippurate in a urine sample taken from the subject. More preferably, the in vitro classification method is based on the in vitro determination of the levels of at least citrate, creatinine, mannitol, hippurate and glucose in a urine sample taken from the subject. Still more preferably, the in vitro classification method is based on the in vitro determination of the levels of at least citrate, creatinine, mannitol, hippurate, glucose, phenylalanine, creatine and 2-Aminoadipic Acid in a urine sample taken from the subject.

A preferred embodiment of the first aspect the invention, relates to a method to classify a subject in need thereof, between tuberculosis patients versus HC subjects which comprises determining in a urine sample of the subject the levels of at least citrate and creatinine and comparing the levels of said markers with respect to the levels of the same markers in a HC or with respect to the reference value ranges for the biomarkers for a HC, wherein the subject is classified as suffering from tuberculosis if different levels of the biomarkers compared to the reference value ranges for the biomarkers for a HC indicate that the subject has tuberculosis.

A preferred embodiment of the first aspect of the invention, comprises determining in a urine sample of the subject the levels of at least citrate, creatinine and hippurate and comparing the levels of said markers with respect to the levels of the same markers in a HC or with respect to the reference value ranges for the biomarkers for a HC, wherein the subject is classified as suffering from tuberculosis if different levels of the biomarkers compared to the reference value ranges for the biomarkers for a HC indicate that the subject has tuberculosis.

A further preferred embodiment of the first aspect of the invention, comprises determining in a urine sample of the subject the levels of at least citrate, creatinine, mannitol and hippurate and comparing the levels of said markers with respect to the levels of the same markers in a HC or with respect to the reference value ranges for the biomarkers for a HC, wherein the subject is classified as suffering from tuberculosis if different levels of the biomarkers compared to the reference value ranges for the biomarkers for a HC indicate that the subject has tuberculosis.

A still further preferred embodiment of the first aspect of the invention, comprises determining in a urine sample of the subject the levels of at least citrate, creatinine, mannitol, hippurate and glucose and comparing the levels of said markers with respect to the levels of the same markers in a HC or with respect to the reference value ranges for the biomarkers for a HC, wherein the subject is classified as suffering from tuberculosis if different levels of the biomarkers compared to the reference value ranges for the biomarkers for a HC indicate that the subject has tuberculosis.

A still further preferred embodiment of the first aspect of the invention, comprises determining in a urine sample of the subject the levels of at least citrate, creatinine, mannitol, hippurate, glucose, phenylalanine, creatine and 2-Aminoadipic Acid and comparing the levels of said markers with respect to the levels of the same markers in a HC or with respect to the reference value ranges for the biomarkers for a HC, wherein the subject is classified as suffering from tuberculosis if different levels of the biomarkers compared to the reference value ranges for the biomarkers for a HC indicate that the subject has tuberculosis.

It is noted that the first classification method of the invention aids in the diagnosis of the subject and therefore, in a preferred embodiment, the first classification method of the invention aids in the diagnosis of a subject in need thereof, in particular aids in determining whether a subject suffers or not from a M. tuberculosis infection (from hereinafter “first diagnosis method of the invention). The term “diagnosis”, as used herein, refers both to the process of attempting to determine and/or identify a possible disease in a subject, i.e. the diagnostic procedure, and to the opinion reached by this process, i.e. the diagnostic opinion. As such, it can also be regarded as an attempt at classification of an individual's condition into separate and distinct categories (such as predicting the “increasing risk” of suffering a disease, meaning “increasing risk” as an increased chance of developing or acquiring a disease compared with a normal individual) that allow medical decisions about treatment and prognosis to be made. It is to be understood that the method, in a preferred embodiment, is a method carried out in vitro, i.e. not practiced on the human or animal body. In particular, the diagnosis to determine tuberculosis patients, relates to the capacity to identify and classify tuberculosis patients. This diagnosis, as it is understood by a person skilled in the art does not claim to be correct in 100% of the analyzed samples. However, it requires that a statistically significant amount of the analyzed samples are classified correctly. The amount that is statistically significant can be established by a person skilled in the art by means of using different statistical tools; illustrative, non-limiting examples of said statistical tools include determining confidence intervals, determining the p-value, the Chi-Square test discriminating functions, etc. Preferred confidence intervals are at least 90%, at least 97%, at least 98%, at least 99%. The p-values are, preferably less than 0.1, less than 0.05, less than 0.01, less than 0.005 or less than 0.0001. The teachings of the present invention preferably allow correctly diagnosing in at least 60%, in at least 70%, in at least 80%, or in at least 90% of the subjects of a determining group or population analyzed.

The first diagnostic method of the invention comprises comparing the level(s) of the metabolic marker(s) identified above, with a reference value. The term “reference value”, as used herein, relates to a predetermined criteria used as a reference for evaluating the values or data obtained from the samples collected from a subject. The reference value or reference level can be an absolute value, a relative value, a value that has an upper or a lower limit, a range of values, an average value, a median value, a mean value, or a value as compared to a particular control or baseline value. A reference value can be based on an individual sample value or can be based on a large number of samples, such as from population of subjects of the chronological age matched group, or based on a pool of samples including or excluding the sample to be tested.

In the context of the present invention, the terms “subject”, “patient” or “individual” are used herein interchangeably to refer to all the animals classified as mammals and includes but is not limited to domestic and farm animals, primates and humans, for example, human beings, non-human primates, cows, horses, pigs, sheep, goats, dogs, cats, or rodents. Preferably, the subject is a male or female human being of any age or race.

In the context of the present invention, the term “metabolic marker” or “metabolite” or “biomarker”, are used herein interchangeably to refers to small molecule compounds, such as substrates for enzymes of metabolic pathways, intermediates of such pathways or the products obtained by a metabolic pathway, the occurrence or amount of which is characteristic for a specific situation, for example tuberculosis. The metabolic markers useful for the first diagnostic method of the invention are those defined in table 2. Table 2 contains the abbreviated common names of the metabolites. The metabolic markers of table 2 are intended to refer to any isomer thereof, including structural and geometric isomers. The term “structural isomer”, as used herein, refers to any of two or more chemical compounds, having the same molecular formula but different structural formulas. The term “geometric isomer” or “stereoisomer” as used herein refers to two or more compounds which contain the same number and types of atoms, and bonds (i.e., the connectivity between atoms is the same), but which have different spatial arrangements of the atoms, for example cis and trans isomers of a double bond, enantiomers, and diastereomers. The abbreviated common name of the amino acid or protein corresponds to the Amino Acid name or Protein to which it belongs followed by an accession number described in the Human Metabolome Database HMDB (http://www.hmdb.ca).

In a preferred embodiment the first diagnostic method of the invention further comprises confirming the diagnosis of tuberculosis by means of the clinical examination of the patient.

In the context of the present invention, the term “level” or “presence”, as used herein, refers to the quantity of a biomarker detectable in a sample. Techniques to assay levels of individual biomarkers from test samples are well known to the skilled technician, and the invention is not limited by the means by which the components are assessed. In one embodiment, levels of the individual components of the metabolomic profile include, without limitation, refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry and IR combined with mass spectrometry. Preferably, levels of the individual components of the biomarker profile are assessed using a proton NMR spectrum.

Therefore, a further preferred embodiment of the first aspect of the invention, the method is carried out by determining a measure of any of the subsets of biomarkers identified in the first aspect of the invention or in any of its preferred embodiments, in a urine biological sample, using +/−0.02 ppm, the biomarker peak regions identified in table 1 of a proton NMR high field spectrum for each biomarker.

Another preferred embodiment of the first aspect of the invention, the method is carried out by determining a measure of any of the subsets of biomarkers identified in the first aspect of the invention or in any of its preferred embodiments, in a urine biological sample, using+1-0.02 ppm, the biomarker peak regions identified in table 5 of a proton NMR low field spectrum for each biomarker.

In addition, it is herein noted that by using the selected chemical shifts shown in table 1 and 5, in tuberculosis vs Healthy Controls a TB predictive value of 100% is obtained. Therefore, a second aspect of the invention, refers to a method of a method to classify a subject in need thereof, between tuberculosis patients versus HC which comprises determining in a urine sample of the subject the presence and level of, +/−0.02 ppm, the selected chemical shifts shown in table 1 (high field) or 5 (low field) and comparing the presence and level of said chemical shifts with respect to the presence and level of said chemical shifts in a HC, wherein the subject is classified as suffering from tuberculosis if differences in the presence or level of said chemical shifts compared to the reference values for HC indicate that the subject has tuberculosis.

It is further noted that in the context of the present invention, the assessment of the levels of the individual components can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing. In the context of the present invention, to assess levels of the individual components of the subject, a urine sample is taken from the subject. The sample may or may not processed prior assaying levels of the components of the metabolic profile. The sample may or may not be stored, e.g., frozen, prior to processing or analysis. Once the sample has been processed, the first method of the invention involves the determination of the levels of the biomarker in the sample. The expression “determining the levels of the biomarker”, as used herein, refers to ascertaining the absolute or relative amount or concentration of the biomarker in the sample. There are many ways to collect quantitative or relational data on biomarkers or metabolites, and the analytical methodology does not affect the utility of metabolite concentrations in assessing a diagnosis. Suitable methods for determining the levels of a given metabolite were already indicated above.
Urine biomarker profile for identifying and classifying tuberculosis patients versus patients affected by respiratory infections caused by S. pneumoniae.

The authors of the present invention have further determined that by using the different subsets of metabolites identified below, and preferably applying PLS-DA, the following discrimination results were obtained for tuberculosis patients versus patients affected by respiratory infections caused by S. pneumoniae:
2-Aminoadipic Acid and creatinine: TB predictive value: 61.29 SD=14.33 Sensitivity=63.24%(10.75) Specificity=79.81% (6.30)
2-Aminoadipic Acid, creatinine, and phenylalanine: TB predictive value: 67.29 SD=11.70 Sensitivity=65.40%(11.72) Specificity=81.33% (5.47)
Citrate, creatinine, mannitol, hippurate, glucose, Phenylalanine, Creatine and 2-Aminoadipic Acid. TB predictive value: 60% SD (13.13%); Sensitivity: 65.91% SD(12.12%); Specifity:79.60% SD(5.84%).

Therefore, a third aspect the invention relates to an in vitro method to classify a subject in need thereof, between tuberculosis patients versus patients affected by respiratory infections caused by S. pneumoniae (from hereinafter “second classification method of the invention”), that comprises the in vitro determination of the levels of at least 2-Aminoadipic Acid and creatinine in a urine sample. Preferably, the in vitro classification method is based on the in vitro determination of the levels of at least 2-Aminoadipic Acid, creatinine, and phenylalanine in a urine sample. More preferably, the in vitro classification method is based on the in vitro determination of the levels of at least citrate, creatinine, mannitol, hippurate, glucose, phenylalanine, creatine and 2-Aminoadipic Acid in a urine sample.

A further preferred embodiment of the third aspect the invention relates to an in vitro method to classify a subject in need thereof, between tuberculosis patients versus patients affected by respiratory infections caused by S. pneumoniae, which comprises: determining in a urine sample of the subject the levels of at least 2-Aminoadipic Acid and creatinine and comparing the levels of said markers with respect to the levels of the same markers in a patient affected by a respiratory infection caused by S. pneumoniae or M. tuberculosis or with respect to a reference value for these biomarkers, wherein the subject is classified as suffering from tuberculosis on the basis of any significant differences in the levels of the biomarkers compared to the reference value for these biomarkers, or compared to the levels of said markers with respect to the levels of the same markers in a patient affected by a respiratory infection caused by S. pneumoniae or M. tuberculosis

A further preferred embodiment of the third aspect of the invention, comprises determining in a urine sample of the subject the levels of at least 2-Aminoadipic Acid, creatinine, and phenylalanine and comparing the levels of said markers with respect to the levels of the same markers in a patient affected by a respiratory infection caused by S. pneumoniae or M. tuberculosis or with respect to the reference value for these biomarkers, wherein the subject is classified as suffering from tuberculosis on the basis of any significant differences in the levels of the biomarkers compared to the reference value for these biomarkers, or compared to the levels of said markers with respect to the levels of the same markers in a patient affected by a respiratory infection caused by S. pneumoniae or M tuberculosis

In still further preferred embodiment of the third aspect of the invention, comprises determining in a urine sample of the subject the levels of at least citrate, creatinine, mannitol, hippurate, glucose, phenylalanine, creatine and 2-Aminoadipic Acid and comparing the levels of said markers with respect to the levels of the same markers in a patient affected by a respiratory infection caused by S. pneumoniae or M. tuberculosis or with respect to a reference value for these biomarkers, wherein the subject is classified as suffering from tuberculosis on the basis of any significant differences in the levels of the biomarkers compared to the reference value for the biomarkers, or compared to the levels of said markers with respect to the levels of the same markers in a patient affected by a respiratory infection caused by S. pneumoniae or M. tuberculosis.

It is noted that the second classification method of the invention aids in the diagnosis of the subject and therefore, in a further preferred embodiment, the second classification method of the invention aids in the diagnosis of a subject in need thereof (from hereinafter second diagnostic method of the invention) between tuberculosis patients versus patients affected by respiratory infections caused by S. pneumoniae.

The second diagnostic method of the invention comprises comparing the level(s) of the metabolic marker(s) with a reference value. The term “reference value”, as used herein, relates to a predetermined criteria used as a reference for evaluating the values or data obtained from the samples collected from a subject. The reference value or reference level can be an absolute value, a relative value, a value that has an upper or a lower limit, a range of values, an average value, a median value, a mean value, or a value as compared to a particular control or baseline value. A reference value can be based on an individual sample value or can be based on a large number of samples, such as from population of subjects of the chronological age matched group, or based on a pool of samples including or excluding the sample to be tested.

In a preferred embodiment, the second diagnostic method of the invention further comprises confirming the diagnosis of tuberculosis by means of the clinical examination of the patient.

As already indicated, the term “level” or “presence”, as used herein, refers to the quantity of a biomarker detectable in a sample. Techniques to assay levels of individual biomarkers from test samples are well known to the skilled technician, and the invention is not limited by the means by which the components are assessed. In one embodiment, levels of the individual components of the metabolomic profile include, without limitation, refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry and IR combined with mass spectrometry. Preferably, levels of the individual components of the biomarker profile are assessed using a proton NMR spectrum.

Therefore, in a further preferred embodiment of the third aspect of the invention or of the second diagnostic method of the invention, determining a measure of any of the subsets of biomarkers identified in the third aspect of the invention or in any of its preferred embodiments, in a urine biological sample, is performed by identifying+1-0.02 ppm, the biomarker peak regions identified in table 1 of a proton NMR high field spectrum for each biomarker.

In a further preferred embodiment of the third aspect of the invention, determining a measure of any of the subsets of biomarkers identified in the third aspect of the invention or in any of its preferred embodiments, in a urine biological sample, is performed by identifying+1-0.02 ppm, the biomarker peak regions identified in table 5 of a proton NMR low field spectrum for each biomarker.

In addition, it is herein noted that by using the selected chemical shifts shown in table 1 and 5, in tuberculosis vs Healthy Controls a TB predictive value of 100% is obtained. Therefore, a fourth aspect of the invention, refers to a method of a method to classify a subject in need thereof, between between tuberculosis patients versus patients affected by respiratory infections caused by Streptococcus pneumoniae, which comprises determining in a urine sample of the subject the presence and level of, +/−0.02 ppm, the selected chemical shifts shown in table 1 (high field) or 5 (low field) and comparing the presence and level of said chemical shifts with respect to the presence and level of said chemical shifts in a reference value (such as the value in tuberculosis patients or in patients affected by respiratory infections caused by Streptococcus pneumoniae), wherein the subject is classified as suffering from tuberculosis on the basis of any significant differences in the presence or level of said chemical shifts compared to the reference value for the biomarkers, or by comparing the presence or level of said chemical shifts with respect to the presence or levels of said chemical shifts in a patient affected by a respiratory infection caused by Streptococcus pneumoniae or Mycobacterium tuberculosis.

It is further noted that in the context of the present invention, the assessment of the levels of the individual components can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing. In the context of the present invention, to assess levels of the individual components of the subject, a urine sample is taken from the subject. The sample may or may not processed prior assaying levels of the components of the metabolic profile. The sample may or may not be stored, e.g., frozen, prior to processing or analysis. Once the sample has been processed, the first method of the invention involves the determination of the levels of the biomarker in the sample. The expression “determining the levels of the biomarker”, as used herein, refers to ascertaining the absolute or relative amount or concentration of the biomarker in the sample. There are many ways to collect quantitative or relational data on biomarkers or metabolites, and the analytical methodology does not affect the utility of metabolite concentrations in assessing a diagnosis. Suitable methods for determining the levels of a given metabolite were already indicated above.
Urine biomarker profile for identifying and classifying patients affected by respiratory infections caused by S. pneumoniae vs HC.

The authors of the present invention have still further determined that by using the different subsets of metabolites identified below, and preferably applying PLS-DA, the following discrimination results were obtained for patients affected by respiratory infections caused by Streptococcus pneumoniae vs HC:
2-Aminoadipic Acid, citrate, creatinine: RI predictive value: 88.85% SD=5.61 Sensitivity=95.96%(3.98) Specificity=90.52% (4.40).
2-Aminoadipic Acid, citrate, creatinine, mannitol, phenylalanine, hippurate: RI predictive value: 96.23 SD=3.86 Sensitivity=96.47%(3.77) Specificity=96.64% (3.44).
Citrate, creatinine, mannitol, hippurate, glucose, Phenylalanine, Creatine and 2-Aminoadipic Acid. RI predictive value: 92.08% SD (5.40%); Sensitivity: 96.85% SD(3.80%); Specifity:93.18% SD(4.49%).

Therefore, a fifth aspect the invention relates to an in vitro method to classify a subject in need thereof, between patients affected by respiratory infections caused by S. pneumoniae vs HC (from hereinafter “third classification method of the invention”), that comprises the in vitro determination of the levels of at least 2-Aminoadipic Acid, citrate, and creatinine in a urine sample. Preferably, the in vitro classification method is based on the in vitro determination of the levels of at least 2-Aminoadipic Acid, citrate, creatinine, mannitol, phenylalanine, and hippurate in a urine sample. More preferably, the in vitro classification method is based on the in vitro determination of the levels of at least citrate, creatinine, mannitol, hippurate, glucose, phenylalanine, creatine and 2-Aminoadipic Acid in a urine sample.

A further preferred embodiment of the fifth aspect the invention relates to an in vitro method to classify a subject in need thereof, between patients affected by respiratory infections caused by Streptococcus pneumoniae vs HC, which comprises: determining in a urine sample of the subject the levels of at least 2-Aminoadipic Acid, citrate, and creatinine and comparing the levels of said markers with respect to the levels of the same markers in a HC or with respect to a reference value for these biomarkers, wherein the subject is classified as suffering from respiratory infections caused by S. pneumoniae on the basis of any significant differences in the levels of the biomarkers compared to the reference value for these biomarkers, or compared to the levels of said markers with respect to the levels of the same markers in a HC.

A further preferred embodiment of the fifth aspect of the invention, comprises determining in a urine sample of the subject the levels of at least 2-Aminoadipic Acid, citrate, creatinine, mannitol, phenylalanine, and hippurate and comparing the levels of said markers with respect to the levels of the same markers in a HC or with respect to the reference value for these biomarkers, wherein the subject is classified as suffering from respiratory infections caused by S. pneumoniae on the basis of any significant differences in the levels of the biomarkers compared to the reference value for these biomarkers, or compared to the levels of said markers with respect to the levels of the same markers in a HC.

In still further preferred embodiment of the fifth aspect of the invention, comprises determining in a urine sample of the subject the levels of at least citrate, creatinine, mannitol, hippurate, glucose, phenylalanine, creatine and 2-Aminoadipic Acid and comparing the levels of said markers with respect to the levels of the same markers in a HC or with respect to a reference value for these biomarkers, wherein the subject is classified as suffering from respiratory infections caused by S. pneumoniae on the basis of any significant differences in the levels of the biomarkers compared to the reference value for these biomarkers, or compared to the levels of said markers with respect to the levels of the same markers in a HC.

It is noted that the third classification method of the invention aids in the diagnosis of the subject and therefore, in a further preferred embodiment, the third classification method of the invention aids in the diagnosis of a subject in need thereof (from hereinafter third diagnostic method of the invention) between patients affected by respiratory infections caused by S. pneumoniae vs HC.

The third diagnostic method of the invention comprises comparing the level(s) of the metabolic marker(s) with a reference value. The term “reference value”, as used herein, relates to a predetermined criteria used as a reference for evaluating the values or data obtained from the samples collected from a subject. The reference value or reference level can be an absolute value, a relative value, a value that has an upper or a lower limit, a range of values, an average value, a median value, a mean value, or a value as compared to a particular control or baseline value. A reference value can be based on an individual sample value or can be based on a large number of samples, such as from population of subjects of the chronological age matched group, or based on a pool of samples including or excluding the sample to be tested.

In a preferred embodiment, the third diagnostic method of the invention further comprises confirming the diagnosis of respiratory infections caused by S. pneumoniae by means of the clinical examination of the patient and microbiological confirmation (culture).

As already indicated, the term “level” or “presence”, as used herein, refers to the quantity of a biomarker detectable in a sample. Techniques to assay levels of individual biomarkers from test samples are well known to the skilled technician, and the invention is not limited by the means by which the components are assessed. In one embodiment, levels of the individual components of the metabolomic profile include, without limitation, refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry and IR combined with mass spectrometry. Preferably, levels of the individual components of the biomarker profile are assessed using a proton NMR spectrum.

Therefore, in a further preferred embodiment of the fifth aspect of the invention or of the third diagnostic method of the invention, determining a measure of any of the subsets of biomarkers identified in the fifth aspect of the invention or in any of its preferred embodiments, in a urine biological sample, is performed by identifying +/−0.02 ppm, the biomarker peak regions identified in table 1 of a proton NMR high field spectrum for each biomarker.

In a further preferred embodiment of the fifth aspect of the invention or of the third diagnostic method of the invention, determining a measure of any of the subsets of biomarkers identified in the fifth aspect of the invention or in any of its preferred embodiments, in a urine biological sample, is performed by identifying +/−0.02 ppm, the biomarker peak regions identified in table 5 of a proton NMR low field spectrum for each biomarker.
Urine biomarker profile for identifying and classifying tuberculosis patients between 0 and 14 years of age, preferably under 18, versus HC and for identifying and classifying tuberculosis patients between 0 and 14 years of age versus vs patients between 0 and 14 years, preferably under 18, affected by latent TB infection (LTBI).

Children's paucibacillary tuberculosis (TB) and their difficulty to expectorate leads to a low diagnostic sensitivity. Most TB in children are diagnosed by clinical scoring systems limited by the TB clinical presentation. Pediatric TB diagnosis research should be focused on new biomarkers from non-invasive and non-sputum-based samples. Metabolomics has the capacity to obtain a “fingerprint” of the metabolites presents in a biological sample, allowing the study of sets of metabolites affected by host-pathogen interactions and the identification of diagnostic markers. This study aims to identify urine metabolite biomarkers with pediatric TB diagnostic potential. The urine spectra from High-resonance 1H Nuclear Magnetic Resonance (NMR) spectroscopy were obtained in a cross-sectional study of 73 children (0-14 years) screened for suspected TB in a pediatric hospital of Haiti. Enrolled children had a positive Tuberculin Skin Test and were classified as follows: 23 TB, 27 probable TB, 13 unlikely TB, and 10 latent TB infection (LTBI). Among all NMR tested samples, an algorithm was performed with 33 spectra from core groups (23 TB, 10 LTBI). We identified eight metabolites with significant changes between groups and when comparing TB and LTBI, we achieved a specificity and sensitivity of 95.49% and 89.90%, respectively. Moreover, when including probable TB and unlikely TB in the algorithm, we classified 92.65% and 53.85%, respectively as TB.

The authors of the present invention have thus determined that by using the subset of metabolites identified below, and preferably applying PLS-DA, the following discrimination results were obtained for patients between 0 and 14 years, preferably under 18, affected by respiratory infections caused by M. tuberculosis (TB) vs patients between 0 and 14 years affected by latent TB infection (LTBI):
Citrate, creatinine, mannitol, hippurate, glucose, Phenylalanine, Creatine and 2-Aminoadipic Acid.
   Active TB vs Non infected individuals (HC): 90.64% (sd 6.38). Sensitivity (95.29%, sd 4.56%), Specificity (91.42%, sd 5.60%).
   Active TB vs LTBI 94.91% (sd 5.53). Sensitivity (95.49%, sd 4.36%), Specificity (89.90%, sd 9.01%).
   LTBI vs Non infected (HC) 78.80% (12.65). Sensitivity (100%), Specificity (91.46%, sd 4.84%).

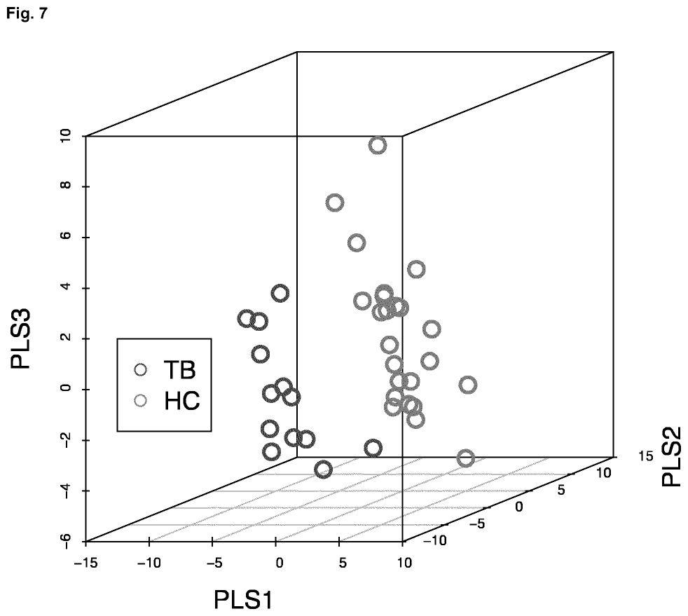

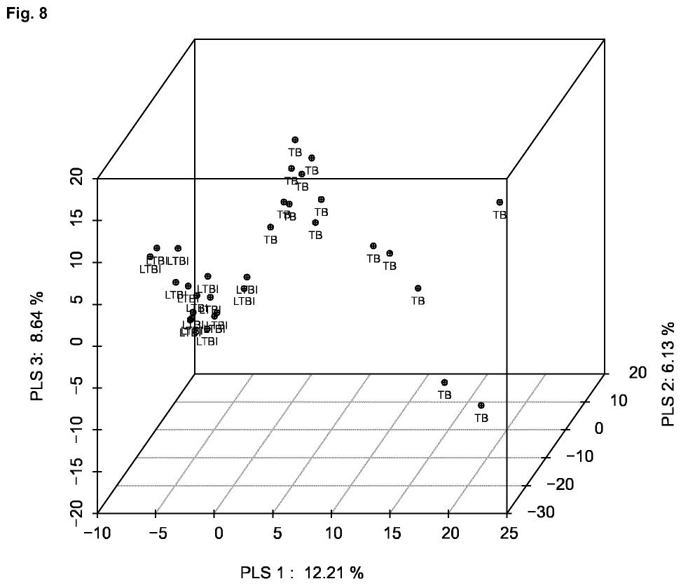

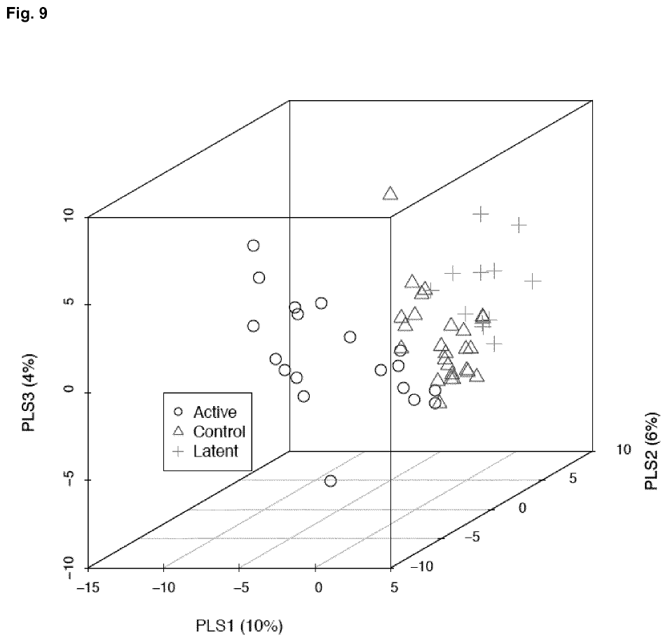

Results are illustrated in FIGS. 7 to 9. These figures show how using the algorithm based in the eight metabolites described above, it is possible to clearly distinguish between children diagnosed of TB from heathy controls individuals (FIG. 7), between children with TB from children diagnosed of latent tuberculosis infection (LTBI) (FIG. 8). In FIG. 9 we can see how we can cluster the patients with active TB from the children with latent TB infection from the patients without latent TB infection. This is especially relevant, because in children the diagnosis of active TB is difficult. Children, especially the youngest ones, are unable to produce sputum, and therefore it is not possible to obtain a diagnosis of active TB, such diagnosis thus having to rely on the epidemiological context of exposure to an adult with smear-positive active TB, and/or TST or IGRA positive, that only gives a presumptive diagnosis of active TB, many times non/distinguishable from LTBI.

Therefore, a sixth aspect the invention relates to an in vitro method to classify a subject between 0 and 14 years of age, preferably below 18 years of age, in need thereof, between i) patients between 0 and 14 years, preferably below 18 years of age, affected by respiratory infections caused by M. tuberculosis (TB) vs ii) patients between 0 and 14 years, preferably below 18 years of age, affected by latent TB infection (LTBI) and optionally vs iii) patients between 0 and 14 years, preferably below 18 years of age, without latent TB infection and without active TB infection (from hereinafter “fourth classification method of the invention”), that comprises the in vitro determination of the levels of at least 2-Aminoadipic Acid, citrate, and creatinine in a urine sample. Preferably, the in vitro classification method is based on the in vitro determination of the levels of at least 2-Aminoadipic Acid, citrate, creatinine, mannitol, phenylalanine, and hippurate in a urine sample. More preferably, the in vitro classification method is based on the in vitro determination of the levels of at least citrate, creatinine, mannitol, hippurate, glucose, phenylalanine, creatine and 2-Aminoadipic Acid in a urine sample.

In the context of the present invention, TB cases or active TB cases are defined as TB confirmed relevant signs and symptoms and microbiologic confirmation of M. tuberculosis-, and TB unconfirmed—patients without bacteriological confirmation but with relevant signs and symptoms, positive TST and/or QFT-GIT, radiological findings suggestive of TB, known TB contact, and clinical response anti-TB treatment. Depending on TB location, active TB cases can be categorized as extra/thoracic TB and intrathoracic TB cases. Within the intrathoracic TB group, patients who present mediastinal lymphadenopathy without lung involvement (mediastinal TB) were subgrouped. LTBI cases are defined as documented patients with TB exposure, positive TST and QFT-GIT, normal chest radiographs, and no clinical signs of TB development in the last 6 months from the diagnosis. Healthy individuals (HC) were classified as infected or uninfected based on the results obtained by the tuberculin test and/or IGRAs. The tuberculous patients were all diagnosed microbiologically by isolation of M. tuberculosis from the clinical samples.

A further preferred embodiment of the sixth aspect of the invention, comprises determining in a urine sample of the subject the levels of at least 2-Aminoadipic Acid, citrate, creatinine, mannitol, phenylalanine, and hippurate and comparing the levels of said markers with respect to the reference value for these biomarkers, wherein the subject is classified as suffering from or having i) active TB from ii) LTBI and optionally from those iii) not having or suffering from latent TB infection and or from active TB infection, on the basis of any significant differences in the levels of the biomarkers compared to the reference value.

In still further preferred embodiment of the sixth aspect of the invention, comprises determining in a urine sample of the subject the levels of at least citrate, creatinine, mannitol, hippurate, glucose, phenylalanine, creatine and 2-Aminoadipic Acid and comparing the levels of said markers with respect to a reference value for these biomarkers, wherein the subject is classified as suffering from or having i) active TB from ii) LTBI and optionally from those iii) not having or suffering from latent TB infection and or from active TB infection, on the basis of any significant differences in the levels of the biomarkers compared to the reference value.

It is noted that the fourth classification method of the invention aids in the diagnosis of the subject and therefore, in a further preferred embodiment, the fourth classification method of the invention aids in the diagnosis of a subject in need thereof (from hereinafter fourth diagnostic method of the invention) between patients affected by active TB vs LTBI.

The fourth diagnostic method of the invention comprises comparing the level(s) of the metabolic marker(s) with a reference value. The term “reference value”, as used herein, relates to a predetermined criteria used as a reference for evaluating the values or data obtained from the samples collected from a subject. The reference value or reference level can be an absolute value, a relative value, a value that has an upper or a lower limit, a range of values, an average value, a median value, a mean value, or a value as compared to a particular control or baseline value. A reference value can be based on an individual sample value or can be based on a large number of samples, such as from population of subjects of the chronological age matched group, or based on a pool of samples including or excluding the sample to be tested.

As already indicated, the term “level” or “presence”, as used herein, refers to the quantity of a biomarker detectable in a sample. Techniques to assay levels of individual biomarkers from test samples are well known to the skilled technician, and the invention is not limited by the means by which the components are assessed. In one embodiment, levels of the individual components of the metabolomic profile include, without limitation, refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry and IR combined with mass spectrometry. Preferably, levels of the individual components of the biomarker profile are assessed using a proton NMR spectrum.

Therefore, in a further preferred embodiment of the sixth aspect of the invention or of the fourth diagnostic method of the invention, determining a measure of any of the subsets of biomarkers identified in the sixth aspect of the invention or in any of its preferred embodiments, in a urine biological sample, is performed by identifying+1-0.02 ppm, the biomarker peak regions identified in table 1 of a proton NMR high field spectrum for each biomarker.

In a further preferred embodiment of the sixth aspect of the invention or of the fourth diagnostic method of the invention, determining a measure of any of the subsets of biomarkers identified in the sixth aspect of the invention or in any of its preferred embodiments, in a urine biological sample, is performed by identifying +/−0.02 ppm, the biomarker peak regions identified in table 5 of a proton NMR low field spectrum for each biomarker.
Urine biomarker profile for identifying and classifying adult tuberculosis patients versus vs adult patients affected by latent TB infection (LTBI).

A total of 147 patients have been studied so far, including a group of healthy controls without evidence of tuberculosis infection (n=31), a group of healthy individuals with tuberculous infection (n=18) and another group with active tuberculosis (n=98).

From each individual, a urine sample was collected, which, after pretreatment, was analyzed by proton NMR spectroscopy (Bruker 700). For each urine sample a one-dimensional spectrum was obtained, whose data was analyzed with the MestreNova software. Subsequently, a multivariate statistical analysis was performed with the Metabonomic software package to identify possible regions susceptible to contain biomarkers for tuberculosis. The identification of these metabolites was studied with the Chenomx software, for which it was necessary to obtain a two-dimensional NMR spectrum from the urine.

| | Relative change in concentration (%) | | | T-Test | | | |
|---|---|---|---|---|---|---|---|
| Metabolite | TB vs HC | TB vs RI | TB vs LTBI | TB-HC | TB-RI | TB vs LTBI | RI-HC |
| Aminoadipic | 82.9 | −67.0 | 17.30 | 9.75E−02 | 1.28E−03 | 6.45E−01 | 3.09E−07 |
| Citrate | −59.6 | 66.2 | −36.72 | 1.14E−04 | 8.81E−02 | 1.62E−02 | 1.36E−08 |
| Creatine | 21.6 | −30.3 | 102.71 | 4.47E−01 | 2.69E−01 | 4.07E−02 | 4.29E−02 |
| Creatinine | −32.3 | 91.6 | −19.36 | 1.32E−03 | 1.15E−03 | 1.79E−01 | 7.65E−12 |
| Glucose | −7.9 | 37.8 | −24.59 | 7.08E−01 | 3.72E−01 | 7.31E−03 | 4.29E−02 |
| Mannitol | 78.8 | −47.7 | 18.13 | 4.36E−02 | 1.07E−01 | 2.62E−01 | 1.03E−03 |
| Phenylalanine | 33.5 | −33.4 | 60.44 | 6.26E−02 | 2.62E−02 | 3.21E−02 | 1.23E−04 |
| Hippurate | −49.6 | 34.8 | −23.32 | 1.32E−02 | 2.70E−01 | 4.72E−01 | 1.47E−04 |

The authors of the present invention have thus determined that by using the different subsets of metabolites identified below, and preferably applying PLS-DA, the following discrimination results were obtained for active tuberculosis patients versus latent tuberculosis patients:
Citrate, creatinine, mannitol and hippurate: TB predictive value: 68.89 SD=11.27 Sensitivity=79.40%(9.09) Specificity=72.95% (7.80).
Citrate, creatinine, mannitol, hippurate and glucose: TB predictive value: 74.11 SD=11.71 Sensitivity=79.69%(8.94) Specificity=76.36% (9.08).
Citrate, creatinine, mannitol, hippurate, glucose, Phenylalanine, Creatine and 2-Aminoadipic Acid. TB predictive value: 74.22% SD(12.01%); Sensitivity: 81.84% SD(8.73%); Specifity: 77.08% SD(9.09%).

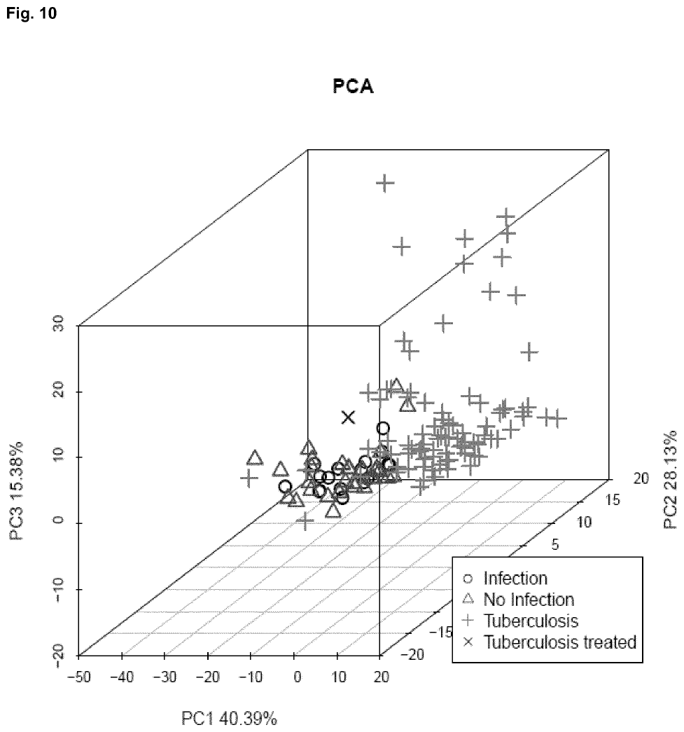

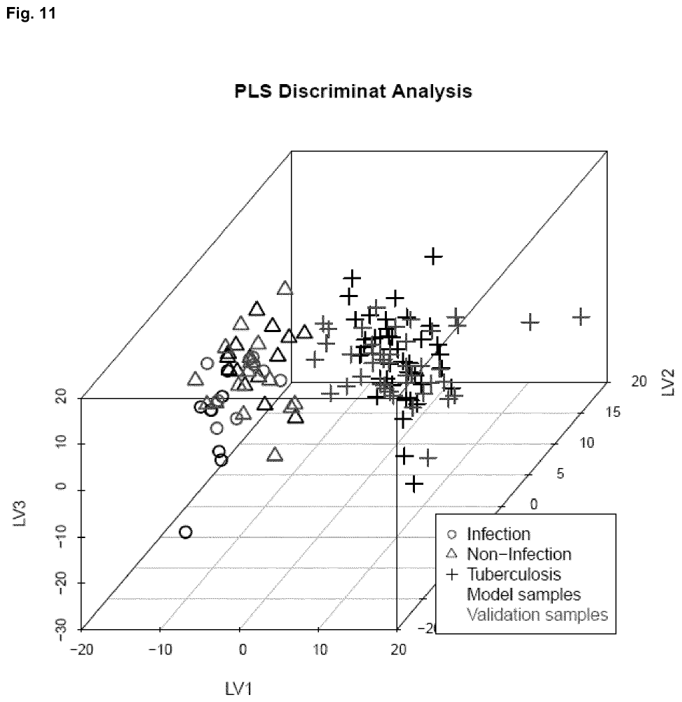

The results are further illustrated in FIGS. 10 to 11, these figures show the multivariate statistical analysis (Analysis of Principal Components and Discriminant Analysis by Partial Least Squares) shows that there are significant differences between the NMR spectra of the group of tuberculous patients with respect to the other two groups studied (p<0.00001) (FIG. 10). The supervised multivariate analysis PLS generates a model that classifies patients with respect to healthy individuals with a sensitivity of 98.7% and a specificity of 95.9% (FIG. 11).

Therefore, a seventh aspect the invention relates to an in vitro method to classify a subject, preferably a human adult, in need thereof, between patients suffering from or having i) active TB from ii) LTBI and optionally from those iii) not having or suffering from latent TB infection and or from active TB infection (from hereinafter “fifth classification method of the invention”), that comprises the in vitro determination of the levels of at least Citrate, creatinine, mannitol and hippurate in a urine sample. Preferably, the in vitro classification method is based on the in vitro determination of the levels of at least Citrate, creatinine, mannitol, hippurate and glucose in a urine sample. More preferably, the in vitro classification method is based on the in vitro determination of the levels of at least Citrate, creatinine, mannitol, hippurate, glucose, Phenylalanine, Creatine and 2-Aminoadipic Acid in a urine sample.

A further preferred embodiment of the seventh aspect the invention relates to an in vitro method to classify a subject in need thereof, between patients suffering from or having i) active TB from ii) LTBI and optionally from those iii) not having or suffering from latent TB infection and or from active TB infection, which comprises: determining in a urine sample of the subject the levels of at least Citrate, creatinine, mannitol and hippurate and comparing the levels of said markers with respect to a reference value for these biomarkers, wherein the subject is classified as suffering from TB or LTBI or not TB or LTBI on the basis of any significant differences in the levels of the biomarkers compared to the reference value.

A further preferred embodiment of the sixth aspect of the invention, comprises determining in a urine sample of the subject the levels of at least Citrate, creatinine, mannitol, hippurate, glucose, Phenylalanine, Creatine and 2-Aminoadipic Acid and comparing the levels of said markers with respect to the reference value for these biomarkers, wherein the subject is classified as suffering from TB or LTBI or not TB or LTBI on the basis of any significant differences in the levels of the biomarkers compared to the reference value.

It is noted that the fifth classification method of the invention aids in the diagnosis of the subject and therefore, in a further preferred embodiment, the fifth classification method of the invention aids in the diagnosis of a subject in need thereof (from hereinafter fifth diagnostic method of the invention) between patients affected by TB vs LTBI.

The fifth diagnostic method of the invention comprises comparing the level(s) of the metabolic marker(s) with a reference value. The term “reference value”, as used herein, relates to a predetermined criteria used as a reference for evaluating the values or data obtained from the samples collected from a subject. The reference value or reference level can be an absolute value, a relative value, a value that has an upper or a lower limit, a range of values, an average value, a median value, a mean value, or a value as compared to a particular control or baseline value. A reference value can be based on an individual sample value or can be based on a large number of samples, such as from population of subjects of the chronological age matched group, or based on a pool of samples including or excluding the sample to be tested.

As already indicated, the term “level” or “presence”, as used herein, refers to the quantity of a biomarker detectable in a sample. Techniques to assay levels of individual biomarkers from test samples are well known to the skilled technician, and the invention is not limited by the means by which the components are assessed. In one embodiment, levels of the individual components of the metabolomic profile include, without limitation, refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry and IR combined with mass spectrometry. Preferably, levels of the individual components of the biomarker profile are assessed using a proton NMR spectrum.

Therefore, in a further preferred embodiment of the seventh aspect of the invention or of the fifth diagnostic method of the invention, determining a measure of any of the subsets of biomarkers identified in the sixth aspect of the invention or in any of its preferred embodiments, in a urine biological sample, is performed by identifying +/−0.02 ppm, the biomarker peak regions identified in table 1 of a proton NMR high field spectrum for each biomarker.

In a further preferred embodiment of the seventh aspect of the invention or of the ffith diagnostic method of the invention, determining a measure of any of the subsets of biomarkers identified in the seventh aspect of the invention or in any of its preferred embodiments, in a urine biological sample, is performed by identifying +/−0.02 ppm, the biomarker peak regions identified in table 5 of a proton NMR low field spectrum for each biomarker.

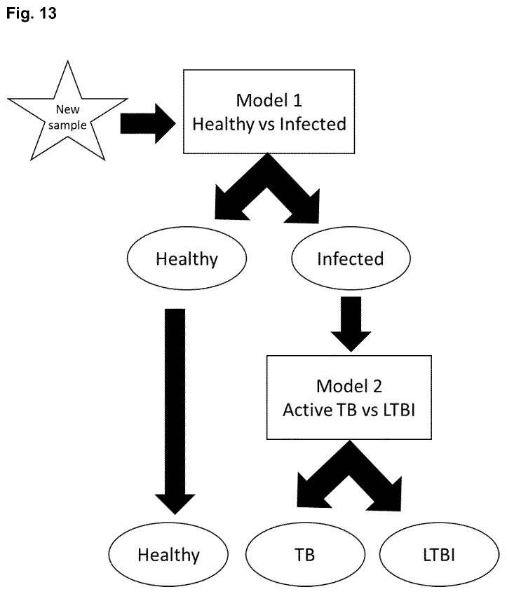

In the context of the present invention, to classify a subject, preferably a human subject independently of its age, in need thereof, between subjects suffering from or having i) active TB from ii) LTBI and optionally from those iii) not having or suffering from latent TB infection and or from active TB infection, the following, exemplary and non-limited, methodology can be used. In this sense, based on the characteristic metabolic profile of active tuberculosis, latent tuberculosis and healthy subjects identified in low field NMR spectra (see examples), two classificatory models were developed by the authors of the present invention and trained for Tuberculosis diagnosis and stratification: (Model-1) Healthy vs TB infected subjects (Active TB and Latent TB); (Model-2) TB vs LTBI subjects. Classificatory models were based on the modification of PLS-DA proposed by Ding and Gentleman [Ding B, Gentleman R, (2005) Classification Using Generalized Partial Least Squares. Journal of Computational and Graphical Statistics 14: 280-298] (tolerance for convergence=1 e10-3, maximum number of iterations allowed=100). Three PLS components were used for classification in each model. New samples classifications were perform as indicated in the flow chart of FIG. 13. As test, 34 samples (HC=14; TB=11; LTBI=9) were classified between groups (Table 1). The results show a high accuracy in tuberculosis stratification following the 8 metabolite signature identified above.
Active Tuberculosis classification: 90,91% Sensitivity: 83,33%; Specifity: 95,45%.
Latent Tuberculosis classification: 77,78% Sensitivity: 100%; Specifity: 91,67%.

TABLE 1
Classification result of the new samples
introduced on classification model.

| Sample # | Sample code | Class | Model 1 | Model 2 | Result |
|---|---|---|---|---|---|
| 16 | DR1216 | HC | HC | | HC |
| 17 | DR1242 | HC | HC | | HC |
| 18 | DR1246 | HC | HC | | HC |
| 19 | DR1158 | HC | Infected | TB | TB |
| 20 | 270 | HC | HC | | HC |
| 21 | 662 | HC | HC | | HC |
| 22 | 893 | HC | HC | | HC |
| 23 | RIR | HC | HC | | HC |
| 24 | 3480 | HC | HC | | HC |
| 25 | CMJ | HC | Infected | TB | TB |
| 26 | 3905 | HC | HC | | HC |
| 27 | 3614 | HC | HC | | HC |
| 28 | MIMB | HC | HC | | HC |
| 29 | DR1241 | HC | HC | | HC |
| 75 | PED72 | TB | Infected | TB | TB |
| 76 | ERJ | TB | Infected | TB | TB |
| 77 | AA | TB | Infected | TB | TB |
| 78 | DR1414 | TB | Infected | TB | TB |
| 79 | U00002 | TB | HC | | HC |
| 80 | U00056 | TB | Infected | TB | TB |
| 81 | U00380 | TB | Infected | TB | TB |
| 82 | U00393 | TB | Infected | TB | TB |
| 83 | Met | TB | Infected | TB | TB |
| 84 | U00028 | TB | Infected | TB | TB |
| 85 | FSMA | TB | Infected | TB | TB |
| 58 | 409 | LTBI | Infected | TB | TB |
| 59 | 410 | LTBI | Infected | LTBI | LTBI |
| 60 | 411 | LTBI | Infected | LTBI | LTBI |
| 61 | 412 | LTBI | HC | | HC |
| 62 | 413 | LTBI | Infected | LTBI | LTBI |
| 63 | 414 | LTBI | Infected | LTBI | LTBI |
| 64 | 415 | LTBI | Infected | LTBI | LTBI |
| 65 | 416 | LTBI | HC | | HC |
| 66 | 417 | LTBI | Infected | LTBI | LTBI |

Method for determining the efficacy of a therapy for tuberculosis

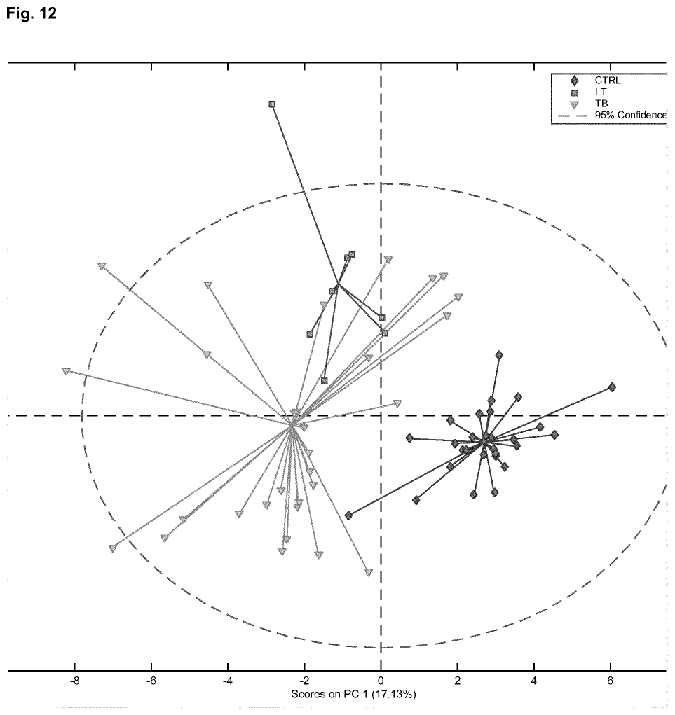

As clearly indicated in FIG. 12 we can see that when we evaluate the metabolome signature (Citrate, creatinine, mannitol, hippurate, glucose, Phenylalanine, Creatine and 2-Aminoadipic Acid) in patients who have been in treatment for more than two months (LT), we can see how the metabolism is still more similar to that of TB patients than that of the controls (CTRL), but turning towards a new class. Therefore, we describe how the signature changes over time, as a surrogate marker of treatment response. This is especially relevant because there is no alternative methods, and accurate monitorization of the treatment is a source of generation of drug-resistance, this is specially serious in patients with multi-drug resistance tuberculosis.

In an eight aspect, the invention relates to a method for determining the efficacy of a therapy for tuberculosis or respiratory infections caused by S. pneumoniae or M. tuberculosis, comprising determining in a urine sample of a subject suffering from any of these diseases, and having been treated with said therapy, the level(s) of the urine biomarker profiles of the first to seventh aspects of the invention, wherein
such level(s) with respect to HC or with respect a reference value are indicative that said therapy is effective or not against M. tuberculosis; and/or
such level(s) with respect to HC or with respect a reference value are indicative that said therapy is effective or not against respiratory infections caused by S. pneumoniae.

The term “therapy for tuberculosis (M. tuberculosis) or respiratory infections caused by S. pneumoniae” as used herein, refers to the attempted remediation of a health problem, usually following a diagnosis, or to prevention of the appearance of a health problem. As such, it is not necessarily a cure, i.e. a complete reversion of a disease. Said therapy may or may not be known to have a positive effect on a particular disease. This term includes both therapeutic treatment and prophylactic or preventative measures, in which the object is to prevent or stop (reduce) an undesired physiological change or disorder. For the purpose of this invention, beneficial or desired clinical results include, without limitation, relieving symptoms, reducing the spread of the disease, stabilizing pathological state (specifically not worsening), slowing down or stopping the progression of the disease, improving or mitigating the pathological state and remission (both partial and complete), both detectable and undetectable. It can also involve prolonging survival, disease free survival and symptom free survival, in comparison with the expected survival if treatment is not received. Those subjects needing treatment include those subjects already suffering the condition or disorder, as well as those with the tendency to suffer the condition or disorder or those in which the condition or disorder must be prevented.

In a particular embodiment, the determination of the level of the one or more metabolic markers is carried out by mass spectrometry or by using a proton NMR spectrum.
Method for monitoring the progression of tuberculosis or respiratory infections caused by S. pneumoniae

In a ninth aspect, the invention relates to a method for monitoring the progression of a subject suffering from M. tuberculosis or respiratory infections caused by S. pneumoniae, comprising determining in a urine sample of a subject suffering from any of these diseases, over the course of a therapy or not, the level(s) of the urine biomarker profiles of the first to seventh aspects of the invention, wherein
such level(s) with respect to a reference value determined in a urine sample from the same subject at an earlier time point are indicative that the tuberculosis condition/disease is progressing; and/or
such level(s) with respect to a reference value determined in a urine sample from the same subject at an earlier time point are indicative that the respiratory infections caused by S. pneumoniae condition/disease is progressing.

The term “monitoring the progression”, as used herein, refers to the determination of the evolution of the disease in a subject diagnosed with tuberculosis or respiratory infections caused by S. pneumoniae, i.e., whether the tuberculosis or respiratory infections caused by S. pneumoniae is worsening or whether it is ameliorating.

The term “progression in the tuberculosis or respiratory infections caused by S. pneumoniae”, as used herein, is understood as a worsening of the disease, i.e., that the disease is progressing to a later stage with respect to a stage at an earlier time point measured. In a particular embodiment, a progression in the tuberculosis or respiratory infections caused by S. pneumoniae means a progression from tuberculosis or respiratory infections caused by Streptococcus pneumoniae in a preclinical stage (non-symptomatic subjects) to tuberculosis or respiratory infections caused by S. pneumoniae with clinical symptoms (symptomatic subjects).
Diagnostic kit

In a final aspect of the invention, the determination of the level of the one or more metabolic markers, to practice any of the aspects of the present invention, can be carried out by any suitable method, such as refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry and IR combined with mass spectrometry. Preferably, levels of the individual components of the biomarker profile are assessed using a proton NMR spectrum. Also preferably, the present invention can be carried out using a test strip which is adapted to receive a sample and detect the urine biomarker profiles of the first to seventh aspects of the invention. According to one embodiment, the test strip comprises a sample addition zone to which a sample may be added; an absorbent zone proximal to the sample addition zone; one or more test zones distal to the sample addition zone, at least one of the test zones including one or more analyte binding agents immobilized therein which are capable of binding to the urine biomarker profiles of the first to seventh aspects of the invention to be detected; and a terminal sample flow zone distal to the one or more test zones, the absorbent zone being positioned relative to the sample addition zone and having an absorption capacity relative to the other zones of the test strip such that a distal diffusion front of a sample added to the sample addition zone diffuses from the sample addition zone to a distal diffusion point within the terminal sample flow zone and then reverses direction and diffuses proximal relative to the one or more test zones.

In another embodiment, a test strip is provided which comprises a sample addition zone to which a sample may be added; an absorbent zone proximal to the sample addition zone; one or more test zones distal to the sample addition zone, at least one of the test zones including one or more analyte binding agents immobilized therein which is capable of binding to the urine biomarker profiles of the first to seventh aspects of the invention to be detected; a terminal sample flow zone distal to the one or more test zones, the absorbent zone being positioned relative to the sample addition zone and having an absorption capacity relative to the other zones of the test strip such that a distal diffusion front of a sample added to the sample addition zone within the predetermined volume range diffuses from the sample addition zone to a distal diffusion point within the terminal sample flow zone and then diffuses proximal relative to the one or more test zones; and a conjugate buffer addition zone distal to the terminal sample flow zone to which a conjugate buffer may be added.

According to the above test strip embodiment, the conjugate buffer addition zone may be positioned relative to the test zones such that conjugate buffer added to the conjugate buffer addition zone at the same time as sample is added to the sample addition zone reaches the distal diffusion point after the distal diffusion front of the sample has diffused to the distal diffusion point and begun diffusing in a proximal direction. The conjugate buffer addition zone may also be positioned relative to the test zones such that conjugate buffer added to the conjugate buffer addition zone at the same time that the sample is added to the sample addition zone reaches the test zones after the distal diffusion front of the sample diffuses proximal relative to the test zones. The conjugate buffer addition zone may also be positioned relative to the test zones such that the conjugate buffer can be added to the test strip before the sample and nevertheless reach the distal diffusion point after the distal diffusion front of the sample has diffused to the distal diffusion zone, reversed direction and begun diffusing in a proximal direction.

According to any of the above test strip embodiments, the test strip may include 1, 2, 3 or more test zones with one or more control binding agents immobilized therein. In one embodiment, the test strip comprises at least a first control zone with one or more control binding agents immobilized therein. Optionally, the test zones further include a second control zone with one or more of the same control binding agents immobilized therein as the first control zone, the first control zone containing a different amount of the control binding agents than the second control zone.

Furthermore and also preferably, the present invention can be carried out using any immunological testing methods which take advantage of the high specificity of antigen-antibody reactions.

In particular, by using a kit suitable for mass spectrometry assay preparation or proton NMR spectrum assay preparation, such kit should preferably deliver the widest range of metabolomic information available from a single targeted assay, covering a large number of key metabolites from main metabolic pathways. This kit should thus quantitatively analyze a large number of metabolites that have already been identified herein as part of key biochemical pathways, providing fundamental data to link changes in the metabolome to biological events. Preferably, such kit should preferably comprise at least one, preferably all, of the following components; a kit's plate, a silicone mat cover for the plate, solvents preferably in sealed glass ampoules, quality controls, standards, a deep well capture plate, a memory stick having a software to link changes in the metabolome to biological events and a user manual.

Yet another aspect of the present invention includes a kit for aiding in the diagnosis of tuberculosis or respiratory infections caused by S. pneumoniae or for the differential diagnosis of tuberculosis vs respiratory infections caused by S. pneumoniae, comprising: biomarker detecting reagents for determining a differential expression level of the specific combinations of biomarkers identified in any of the aspects of the present invention.

In one preferred embodiment of this aspect of the invention, the kit further comprises instructions for use in diagnosing risk for tuberculosis or respiratory infections caused by S. pneumoniae or for the differential diagnosis of tuberculosis vs respiratory infections caused by S. pneumoniae, wherein the instruction comprise step-by-step directions to compare the expression level of the specific combinations of biomarkers identified in any of the aspects of the present invention, when measuring the expression of a urine sample obtained from a subject suspected of having tuberculosis or respiratory infections caused by S. pneumoniae with the expression level of a sample obtained from a normal subject, wherein the normal subject is a healthy subject not suffering from tuberculosis or respiratory infections caused by S. pneumoniae, or with a reference value. In another aspect, the kit further comprises tools, vessels and reagents necessary to obtain urine samples from a subject.

Yet another aspect of the present invention includes a computer program suitable for implementing any of the methods of the present invention. In addition, a device comprising the above mentioned computer program also forms part of the present invention as well as its use for the diagnosis of tuberculosis or respiratory infections caused by S. pneumoniae in a human subject. In this sense, the assignment of a patient into a specific group of patients, such as patients with tuberculosis or respiratory infections caused by S. pneumoniae, by any of the methods of the invention can be done by a computer program, preferably, after introducing the data into said program. Thus, in another preferred embodiment, the step of assigning a patient into a specific group of patients, such as patients with tuberculosis or respiratory infections caused by S. pneumoniae, according to any of the methods described in the present specification, is a computer implemented step wherein the data obtained in the previous steps of the method are inserted in a computer program and the program assigns the patient into one of the groups of patients.

The present invention is further illustrated by the following examples which merely illustrate the invention and do not limit the same.

### EXAMPLE

### Material and Methods

1. NMR High Field Acquisition

Urine samples from patients diagnosed of tuberculosis (TB, n=19), respiratory infections caused by S. pneumoniae (RI, n=25) and healthy controls (HC, n=29) were examined using a Bruker Avance spectrometer operating at 16.4 T. Before NMR acquisition, urine samples were pH adjusted using a 0.2M Phosphate Buffer (pH=7.4) containing 0.3 mM TSP as internal reference. 1D proton NMR spectra were recorded using a NOESY pulse sequence. Standard solvent-suppressed spectra were grouped into 32,000 data points, averaged over 256 acquisitions.

1H-1H total correlated spectroscopy (TOCSY), and gradient-selected heteronuclear single quantum correlation (HSQC) protocols were performed to carry out component assignments. Between consecutive two-dimensional (2D) spectra, a control 1HNMR spectrumwas always measured. No gross degradation was noted in the signals of multiple spectra acquired under the same conditions.

The free induction decay (FID) signals were multiplied by an exponential weight function corresponding to line broadening of 0.3 Hz. Spectra were referenced to the TSP singlet at 0 ppm chemical shift. NMR spectra were data-reduced to equal length integral segments (6=0.04 ppm) and they were normalized to total sum of the spectral regions.

2. NMR Low Field Acquisition

Urine samples from patients diagnosed of tuberculosis (n=18) and healthy controls (n=19) were examined using a Magritek spectrometer operating at 1.4 T. Before NMR acquisition, urine samples were pH adjusted using a 0.2M Phosphate Buffer (pH=7.4) containing 0.3 mM TSP as internal reference. NMR spectra were recorded using a Magritek Spinsolve Ultra 60 MHz. 1D proton NMR spectra were recorded using 1D PRESAT pulse sequence and averaged over 64 acquisitions. The free induction decay (FID) signals were multiplied by an exponential weight function corresponding to line broadening of 0.3 Hz. Spectra were referenced to the TSP singlet at 0 ppm chemical shift. NMR spectra were data-reduced to equal length integral segments (δ=0.04 ppm) and they were normalized to total sum of the spectral regions.

3. Statistical Analysis

Principal Component Analysis (PCA) was applied to identify metabolic differences between groups in High Field NMR spectra. PCAs were centered and Pareto scaled. The potential biomarkers from PCA loading plots were selected by Hotteling's T2 tests (p<0.01).

4. Metabolites Identification:

Resonances determined as significantly different between IDHwt and IDHmut cells were identified using the Human Metabolome Database [26] and Chenomx NMR Suit (rel. 7.7) database and individually integrated for metabolic quantification using the Global Spectral Deconvolution algorithm of MestreNova v. 8.1 (Mestrelab Research S.L., Santiago de Compostela, Spain). Statistical significance was determined using an unpaired Student's t test assuming unequal variance with p<0.05 considered significant.

5. Classificatory Analyses

Using the selected metabolites or chemical shifts, partial least-squares discriminant analyses (PLS-DAs) were developed as classificatory models. We used the algorithm proposed by Ding and Gentleman [34] (tolerance for convergence=1 e10-3, maximum number of iterations allowed=100). The number of PLS components used was chosen by the percentage of variance explained, the R2, and the mean squared error of cross-validation graphics. For training purposes, the classification functions derived from the probability of belonging to each group were computed with a number of random testing subjects. These classification functions were used afterwards to classify the rest of the subjects for internal validation. This process was repeated 100 times with random permutations of the data to reduce type I errors. The percentages of correct classification were calculated as a measure of model performance.

### Results

1. Biomarkers Identification

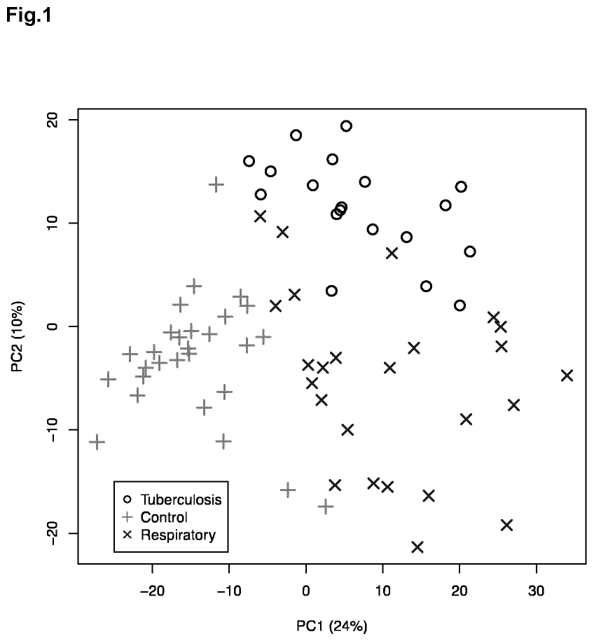

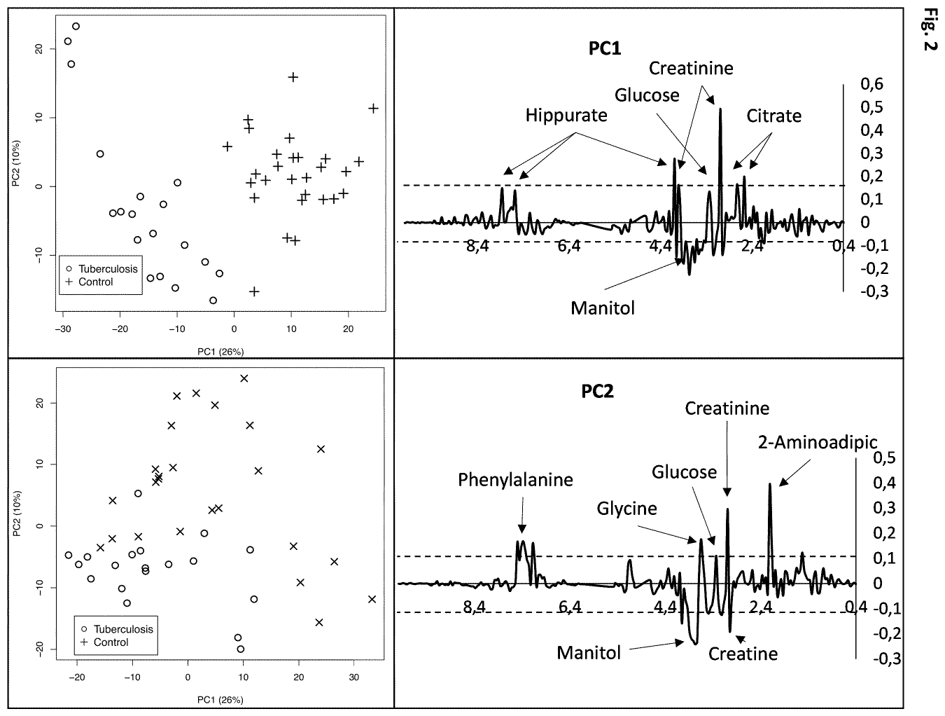

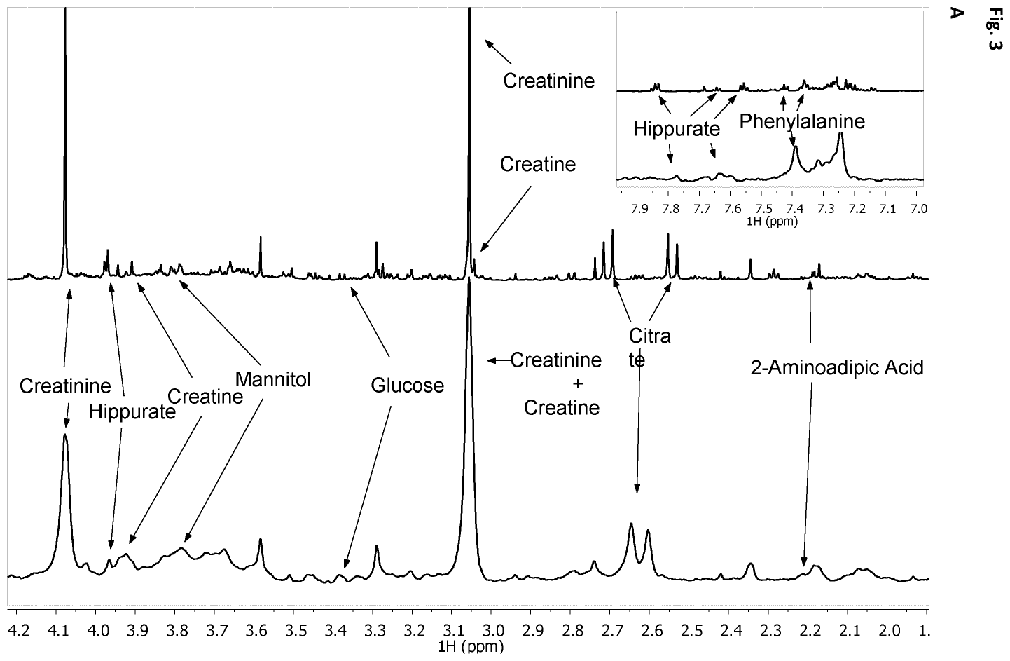

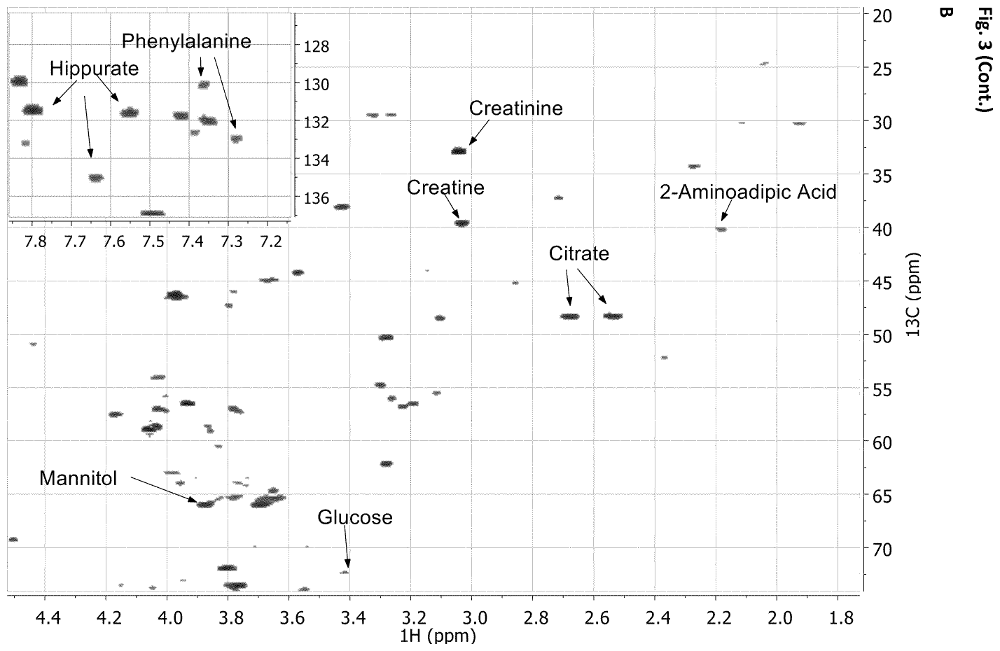

Unsupervised classification studies with PCA were carried out to analyze the differences between spectra from TB, HC and RI urine samples. The urine spectra provided nearly perfect discrimination between TB, HC and RI along the first two principal components (FIG. 1). Individual PCAs were carry out to identify significant differences between TB patients and control groups (FIG. 2, Table 1). The resonances were identified according the Human Metabolome Database [33], and characteristic cross-peaks from 2D ¹H-¹³C spectra to help in unequivocal assignation of these metabolites (FIG. 3-B). Metabolic differences elicited by our method between groups are highlighted in representative High Field and Low Field NMR spectra (FIG. 3-A) and quantified in High Field NMR spectra (table 2). Table 3 shows intensity ratios between significant metabolites. The metabolic intensities were normalized to creatinine signal too (Table 4). The loss of the significance may indicate that all the metabolic changes are correlated, then they provide a single metabolic signature.

2. High Field NMR Classificatory Models

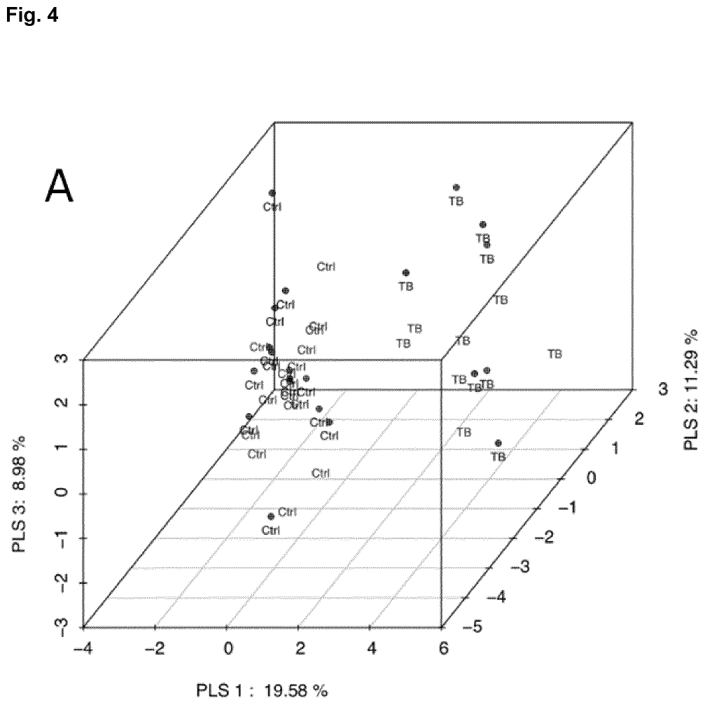

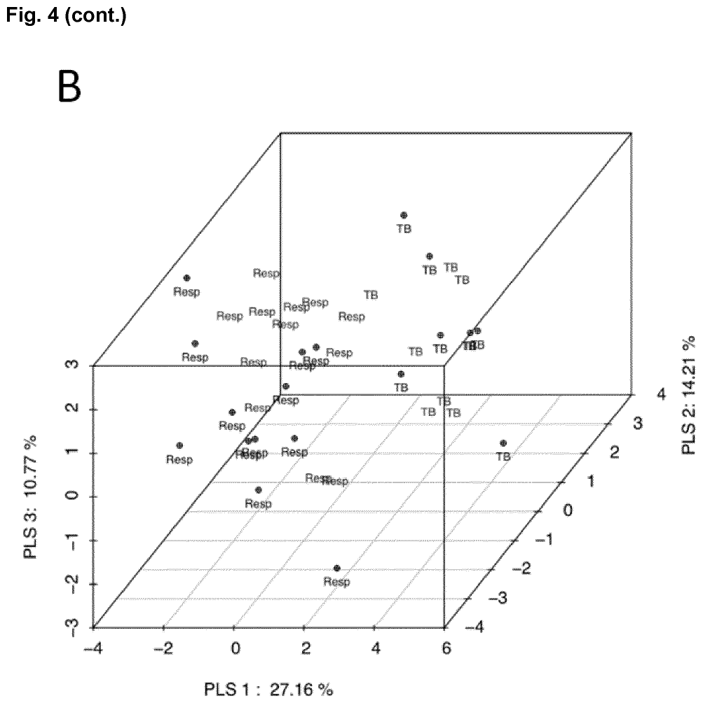

Using the selected chemical shifts (table 1), PLS-DA was applied to discriminate between groups.
Tuberculosis vs Healthy Controls: TB predictive value: 100%; Sensitivity: 100%; Specifity: 100%. FIG. 4-A.
Tuberculosis vs Respiratory Infections: TB predictive value: 100%; Sensitivity: 100%; Specifity: 100%. FIG. 4-B.
Tuberculosis vs Latent Tuberculosis: TB predictive value: 80.56% SD(10.28%); Sensitivity: 93.49% SD(6.25%); Specifity: 83.53% SD(7.68%)

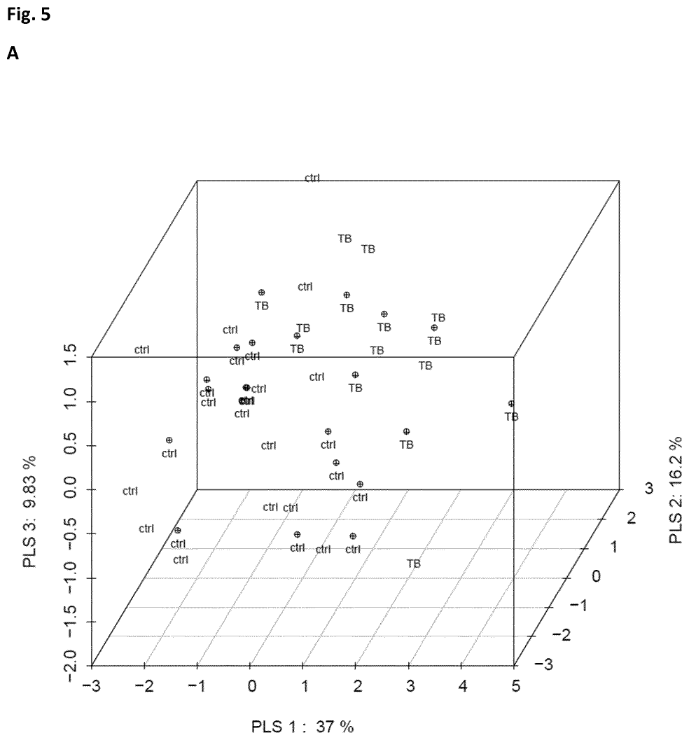

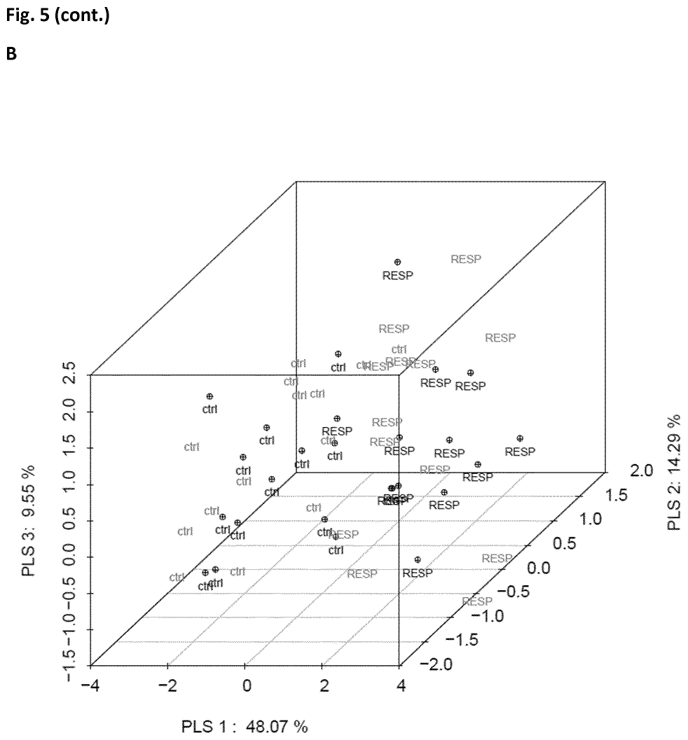

Using the selected metabolites (table 2), PLS-DA was applied to discriminate between groups.
Tuberculosis vs Healthy Controls: TB predictive value: 79.29% SD(3.58%); Sensitivity: 91.22% SD(7.99%); Specifity:90.52% SD(5.05%). FIG. 5-A.
Tuberculosis vs Respiratory Infections: TB predictive value: 60% SD (13.13%); Sensitivity: 65.91% SD(12.12%); Specifity:79.60% SD(5.84%). FIG. 5-B.
Tuberculosis vs Latent Tuberculosis: TB predictive value: 74.22% SD(12.01%); Sensitivity: 81.84% SD(8.73%); Specifity: 77.08% SD(9.09%)

3. Low Field NMR Clasificatory Models

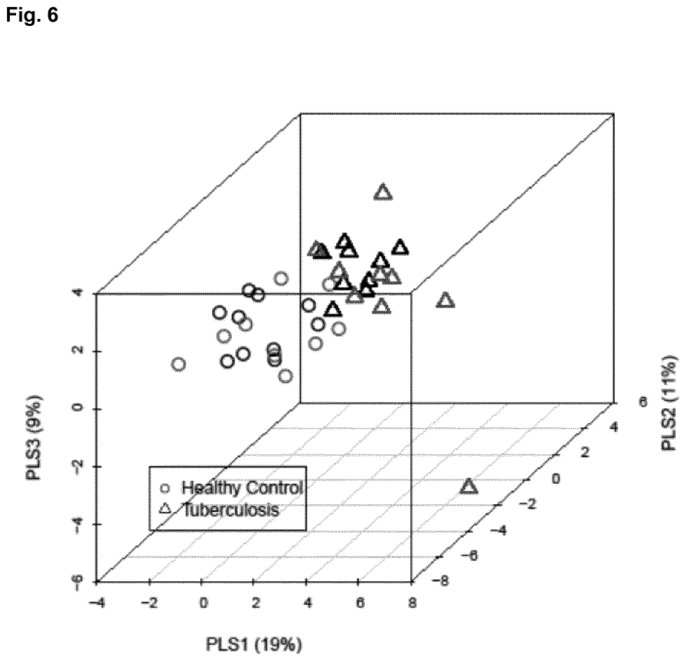

Using the same chemical shifts identified in HF NMR spectra (table 5), PLS-DA was applied to discriminate between groups.
Tuberculosis vs Healthy Controls: TB predictive value: 100%; Sensitivity: 91.12% SD (6.67%); Specifity:100%. FIG. 6.
Tuberculosis vs Respiratory Infections: TB predictive value: 88.22% SD (8.02%); Sensitivity: 81.39% SD(7.48%); Specifity:88.94% SD(7.23%).
Tuberculosis vs Latent Tuberculosis: TB predictive value: 100%; Sensitivity: 88.62% SD(5.93%); Specifity: 100%.

TABLE 2
Summary of significant spectral regions of postulated metabolites and their relative
amounts from Tuberculosis, Respiratory Infection and Healthy Control groups. Intensity
in arbitrary units normalized to sum of all metabolites detected by NMR spectroscopy

| Chemical | | | | Respiratory | | Healthy | | | | |
|---|---|---|---|---|---|---|---|---|---|---|
| Shift region | Prevalent | Tuberculosis | | Infection | | Control | | T-Test | | |
| (ppm) | Metabolite | Mean | SD | Mean | SD | Mean | SD | TB − RI | TB − HC | RI − HC |
| 2.20 | 2-Aminoadipic Acid | 7.18 | 8.74 | 43.42 | 29.73 | 8.38 | 16.81 | 8.05E−05 | 8.05E−01 | 1.15E−05 |
| 2.24 | 2-Aminoadipic Acid | 5.42 | 2.99 | 2.30 | 1.90 | 1.33 | 1.01 | 2.99E−04 | 6.85E−08 | 4.30E−02 |
| 2.60 | Citrate | 1.63 | 1.69 | 1.28 | 2.95 | 0.72 | 1.48 | 6.86E−01 | 8.02E−02 | 2.58E−01 |
| 2.72 | Citrate | 6.70 | 3.98 | 6.16 | 5.01 | 22.40 | 8.95 | 7.34E−01 | 2.25E−07 | 7.32E−11 |
| 2.76 | Citrate | 2.10 | 1.37 | 3.00 | 2.48 | 3.81 | 3.51 | 2.19E−01 | 8.82E−02 | 3.24E−01 |
| 3.08 | Creatine | 39.52 | 22.01 | 61.57 | 34.49 | 145.9 | 47.02 | 3.80E−02 | 7.93E−10 | 2.34E−10 |
| 3.12 | Creatinine | 8.84 | 3.28 | 4.16 | 2.54 | 7.57 | 6.87 | 1.56E−05 | 5.18E−01 | 1.65E−02 |
| 3.16 | Creatinine | 9.12 | 3.96 | 6.00 | 2.95 | 9.26 | 2.47 | 8.08E−03 | 8.94E−01 | 1.10E−04 |
| 3.36 | Glucose | 3.77 | 3.73 | 2.88 | 3.71 | 7.59 | 3.54 | 4.80E−01 | 2.37E−03 | 2.92E−05 |
| 3.40 | Glucose | 4.14 | 3.39 | 1.90 | 2.47 | 1.39 | 2.07 | 2.30E−02 | 2.28E−03 | 4.80E−01 |
| 3.64 | Mannitol | 29.57 | 10.12 | 43.19 | 14.76 | 16.15 | 11.50 | 4.05E−03 | 6.46E−04 | 8.72E−09 |
| 3.69 | Mannitol | 30.66 | 10.26 | 47.76 | 39.15 | 27.44 | 15.11 | 1.19E−01 | 4.78E−01 | 1.21E−02 |
| 3.73 | Mannitol | 22.55 | 7.32 | 32.71 | 29.27 | 15.02 | 8.62 | 2.12E−01 | 7.90E−03 | 3.10E−03 |
| 3.77 | Mannitol | 28.08 | 10.10 | 22.58 | 13.81 | 8.16 | 5.16 | 2.00E−01 | 1.63E−10 | 1.79E−05 |
| 3.81 | Mannitol | 27.10 | 14.03 | 43.13 | 38.96 | 24.12 | 10.92 | 1.47E−01 | 4.54E−01 | 1.19E−02 |
| 3.85 | Mannitol | 21.14 | 10.60 | 28.22 | 29.86 | 19.43 | 10.95 | 3.99E−01 | 6.32E−01 | 1.31E−01 |
| 3.89 | Mannitol | 16.72 | 10.88 | 24.89 | 22.68 | 10.31 | 6.63 | 2.14E−01 | 2.25E−02 | 1.43E−03 |
| 3.93 | Mannitol | 16.88 | 9.37 | 24.76 | 15.66 | 10.06 | 7.06 | 9.48E−02 | 1.18E−02 | 2.42E−05 |
| 3.97 | Mannitol | 23.93 | 8.84 | 15.63 | 18.38 | 28.13 | 14.07 | 1.22E−01 | 3.15E−01 | 4.13E−02 |
| 4.01 | Creatine | 18.67 | 6.38 | 13.86 | 7.59 | 33.24 | 16.71 | 5.24E−02 | 3.22E−03 | 2.55E−06 |
| 4.05 | Creatinine | 36.31 | 25.18 | 13.20 | 6.18 | 19.01 | 7.70 | 8.85E−05 | 1.68E−03 | 1.99E−03 |
| 4.09 | Hippurate | 36.64 | 20.70 | 35.76 | 14.41 | 74.73 | 21.22 | 8.76E−01 | 2.18E−06 | 1.70E−09 |
| 4.13 | Hippurate | 9.20 | 6.66 | 3.04 | 6.01 | 5.46 | 11.33 | 5.42E−03 | 2.63E−01 | 4.48E−01 |
| 4.17 | Hippurate | 7.00 | 2.17 | 2.74 | 1.88 | 3.54 | 1.23 | 1.76E−07 | 6.81E−08 | 1.36E−01 |
| 7.21 | Phenylalanine | 4.54 | 1.39 | 3.52 | 2.71 | 3.63 | 1.65 | 1.98E−01 | 8.48E−02 | 9.22E−01 |
| 7.37 | Phenylalanine | 13.50 | 7.15 | 19.00 | 9.08 | 10.22 | 5.93 | 5.90E−02 | 1.23E−01 | 4.24E−04 |
| 7.41 | Phenylalanine | 7.26 | 3.57 | 9.89 | 6.22 | 7.45 | 6.08 | 1.55E−01 | 9.13E−01 | 2.35E−01 |
| 7.45 | Phenylalanine | 8.01 | 3.28 | 9.52 | 4.89 | 6.64 | 3.20 | 3.10E−01 | 2.03E−01 | 3.86E−02 |
| 7.53 | Hippurate | 4.72 | 4.99 | 2.90 | 1.57 | 4.53 | 2.96 | 9.77E−02 | 8.76E−01 | 1.29E−02 |
| 7.61 | Hippurate | 2.89 | 2.94 | 2.05 | 1.49 | 5.72 | 4.72 | 2.42E−01 | 4.76E−02 | 3.15E−04 |
| 7.89 | Hippurate | 1.62 | 1.87 | 1.73 | 1.74 | 4.63 | 5.08 | 8.58E−01 | 3.88E−02 | 8.26E−03 |

TABLE 3
Quantification of selected metabolites for Diagnostic Model. Intensity in arbitrary
units normalized to sum of all metabolites detected by NMR spectroscopy

| | | | Respiratory | | Healthy | | TB vs | TB vs | | | |
|---|---|---|---|---|---|---|---|---|---|---|---|
| | Tuberculosis | | Infection | | Control | | HC | RI | T-Test | | |
| Metabolite | Mean | SD | Mean | SD | Mean | SD | % | % | TB − RI | TB − HC | RI − HC |
| Aminoadipic | 8.38 | 9.30 | 25.39 | 17.45 | 4.58 | 5.44 | 82.9 | −67.0 | 1.28E−03 | 9.75E−02 | 3.09E−07 |
| Citrate | 13.51 | 9.41 | 8.13 | 9.41 | 33.44 | 16.66 | −59.6 | 66.2 | 8.81E−02 | 1.14E−04 | 1.36E−08 |
| Creatine | 26.85 | 20.61 | 38.51 | 36.88 | 22.08 | 18.75 | 21.6 | −30.3 | 2.69E−01 | 4.47E−01 | 4.29E−02 |
| Creatinine | 109.51 | 51.64 | 57.16 | 41.62 | 161.70 | 44.91 | −32.3 | 91.6 | 1.15E−03 | 1.32E−03 | 7.65E−12 |
| Glucose | 3.71 | 3.78 | 2.70 | 3.25 | 4.03 | 1.81 | −7.9 | 37.8 | 3.72E−01 | 7.08E−01 | 4.29E−02 |
| Mannitol | 20.46 | 18.28 | 39.15 | 41.37 | 11.44 | 10.24 | 78.8 | −47.7 | 1.07E−01 | 4.36E−02 | 1.03E−03 |
| Phenylalanine | 41.03 | 19.88 | 61.59 | 30.68 | 30.74 | 14.95 | 33.5 | −33.4 | 2.62E−02 | 6.26E−02 | 1.23E−04 |
| Hippurate | 5.97 | 5.89 | 4.43 | 2.82 | 11.83 | 7.62 | −49.6 | 34.8 | 2.70E−01 | 1.32E−02 | 1.47E−04 |

TABLE 4
Ratios of selected metabolites for Diagnostic Model

| | Aminoadipic | Citrate | Creatine | Creatinine | Glucose | Mannitol | Phenylalanine | Hippurate |
|---|---|---|---|---|---|---|---|---|
| Aminoadipic | 1.000 | 0.620 | 0.312 | 0.077 | 2.259 | 0.410 | 0.204 | 1.404 |
| Citrate | 1.612 | 1.000 | 0.503 | 0.123 | 3.642 | 0.660 | 0.329 | 2.263 |
| Creatine | 3.204 | 1.987 | 1.000 | 0.245 | 7.237 | 1.312 | 0.654 | 4.497 |
| Creatinine | 13.068 | 8.106 | 4.079 | 1.000 | 29.518 | 5.352 | 2.669 | 18.343 |
| Glucose | 0.443 | 0.275 | 0.138 | 0.034 | 1.000 | 0.181 | 0.090 | 0.621 |
| Mannitol | 2.442 | 1.514 | 0.762 | 0.187 | 5.515 | 1.000 | 0.499 | 3.427 |
| Phenylalanine | 4.896 | 3.037 | 1.528 | 0.375 | 11.059 | 2.005 | 1.000 | 6.873 |
| Hippurate | 0.712 | 0.442 | 0.222 | 0.055 | 1.609 | 0.292 | 0.146 | 1.000 |

TABLE 5
Quantification of selected metabolites for Diagnostic Model.
Intensity in arbitrary units normalized to creatinine signal.

| | | | Respiratory | | Healthy | | TB vs | TB vs | | | |
|---|---|---|---|---|---|---|---|---|---|---|---|
| | Tuberculosis | | Infection | | Control | | HC | RI | T-Test | | |
| Metabolite | Mean | SD | Mean | SD | Mean | SD | % | % | TB − RI | TB − HC | RI − HC |
| Aminoadipic | 0.41 | 1.29 | 2.53 | 5.04 | 0.03 | 0.04 | 1183 | −84.0 | 1.19E−01 | 1.28E−01 | 1.28E−02 |
| Citrate | 0.18 | 0.18 | 0.43 | 0.94 | 0.22 | 0.11 | −18.9 | −58.5 | 3.13E−01 | 3.53E−01 | 9.52E−02 |
| Creatine | 0.56 | 1.10 | 5.16 | 9.84 | 0.17 | 0.17 | 239.3 | −89.1 | 8.10E−02 | 6.65E−02 | 4.94E−03 |
| Glucose | 0.04 | 0.04 | 0.12 | 0.44 | 0.03 | 0.02 | 57.5 | −65.2 | 4.98E−01 | 9.79E−02 | 1.19E−01 |
| Mannitol | 0.77 | 2.28 | 7.17 | 23.22 | 0.09 | 0.10 | 803.0 | −89.2 | 2.97E−01 | 1.15E−01 | 5.78E−02 |
| Phenylalanine | 1.41 | 4.01 | 6.39 | 12.03 | 0.21 | 0.14 | 570.3 | −77.9 | 1.30E−01 | 1.17E−01 | 3.63E−03 |
| Hippurate | 0.17 | 0.45 | 0.43 | 1.01 | 0.08 | 0.05 | 119.8 | −59.6 | 3.63E−01 | 2.76E−01 | 9.12E−02 |

TABLE 6
Summary of significant spectral regions in Low
Field NMR spectra and postulated metabolites

| Chemical Shift | |
|---|---|
| region (ppm) | Prevalent Metabolite |
| 2.20 | 2-Aminoadipic Acid |
| 2.24 | 2-Aminoadipic Acid |
| 2.28 | 2-Aminoadipic Acid |
| 2.60 | Citrate |
| 2.64 | Citrate |
| 2.72 | Citrate |
| 3.04 | Creatine |
| 3.08 | Creatinine |
| 3.12 | Creatinine |
| 3.16 | Creatinine |
| 3.20 | Creatinine |
| 3.36 | Glucose |
| 3.40 | Glucose |
| 3.64 | Mannitol |
| 3.68 | Mannitol |
| 3.72 | Mannitol |
| 3.76 | Mannitol |
| 3.80 | Mannitol |
| 3.84 | Mannitol |
| 3.88 | Mannitol |
| 3.92 | Mannitol |
| 3.96 | Mannitol |
| 4.00 | Creatine |
| 4.04 | Creatinine |
| 4.08 | Hippurate |
| 4.12 | Hippurate |
| 4.16 | Hippurate |
| 7.20 | Phenylalanine |
| 7.24 | Phenylalanine |
| 7.36 | Phenylalanine |
| 7.40 | Phenylalanine |
| 7.44 | Phenylalanine |
| 7.52 | Hippurate |
| 7.56 | Hippurate |
| 7.60 | Hippurate |
| 7.64 | Hippurate |
| 7.88 | Hippurate |
| 7.92 | Hippurate |

1. An in vitro method to classify a subject in need thereof, between subjects suffering from tuberculosis, that is to say infected with M. tuberculosis and suffering the symptomatology of the disease, vs subjects suffering from latent tuberculosis infection, vs subjects not suffering from latent TB infection nor from active TB infection, that comprises the in vitro determination of the levels of at least Citrate, creatinine, mannitol and hippurate in a urine sample taken from the subject.

2. The in vitro method of claim 1, wherein the in vitro classification method is based on the in vitro determination of the levels of at least Citrate, creatinine, mannitol, hippurate and glucose in a urine sample taken from the subject.

3. The in vitro method of claim 1, wherein the in vitro classification method is based on the in vitro determination of the levels of at least Citrate, creatinine, mannitol, hippurate, glucose, Phenylalanine, Creatine and 2-Aminoadipic Acid in a urine sample taken from the subject.

4. The in vitro method of claim 3, wherein the subjects are between 0 and 14 years of age.

5. The in vitro method of claim 1, wherein the in vitro classification method comprises determining in a urine sample of the subject the levels of at least Citrate, creatinine, mannitol and hippurate and comparing the levels of said markers with respect to a reference value for these biomarkers, wherein the subject is classified as suffering from i) active TB or ii) LTBI or ii) not TB or LTBI, on the basis of any significant differences in the levels of the biomarkers compared to the reference value.

6. The in vitro method of claim 2, wherein the in vitro classification method comprises determining in a urine sample of the subject the levels of at least Citrate, creatinine, mannitol, hippurate and glucose and comparing the levels of said markers with respect to a reference value for these biomarkers, wherein the subject is classified as suffering from i) active TB or ii) LTBI or ii) not TB or LTBI, on the basis of any significant differences in the levels of the biomarkers compared to the reference value.

7. The in vitro method of claim 3, wherein the in vitro classification method comprises determining in a urine sample of the subject the levels of at least Citrate, creatinine, mannitol, hippurate, glucose, Phenylalanine, Creatine and 2-Aminoadipic Acid and comparing the levels of said markers with respect to a reference value for these biomarkers, wherein the subject is classified as suffering from i) active TB or ii) LTBI or ii) not TB or LTBI, on the basis of any significant differences in the levels of the biomarkers compared to the reference value.

8. The in vitro method of claim 7, wherein the subjects are between 0 and 14 years of age.

9. The in vitro method of any of the precedent claims, for aiding in the diagnosis of whether a subject suffers or not from a M. tuberculosis infection or from latent tuberculosis infection, and optionally confirming the diagnosis of tuberculosis by means of the clinical examination of the patient.

10. The in vitro method of any of the precedent claims, wherein the in vitro determination of the levels is carried out by using any of the techniques selected from the list consisting of: refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry and IR combined with mass spectrometry.

11. The in vitro method of claim 12, wherein e in vitro determination of the levels is carried out by using a proton NMR spectrum.

12. A method for determining the efficacy of a therapy for tuberculosis or respiratory infections caused by S. pneumoniae, comprising determining in a urine sample of a subject suffering from any of these diseases, and having been treated with said therapy, the level(s) of the biomarkers identified in any of claims 1 to 7, wherein
a. such level(s) with respect to a reference value are indicative that said therapy is effective or not against tuberculosis; and/or
b. such level(s) with respect to a reference value are indicative that said therapy is effective or not against respiratory infections caused by S. pneumoniae.

13. The method of claim 12, wherein the biomarkers identified are the levels of at least Citrate, creatinine, mannitol, hippurate, glucose, Phenylalanine, Creatine and 2-Aminoadipic Acid.

14. A method for monitoring the progression of a subject suffering from tuberculosis or respiratory infections caused by S. pneumoniae, comprising determining in a urine sample of a subject suffering from any of these diseases, over the course of a therapy or not, the level(s) of the biomarkers identified in any of claims 1 to 7, wherein
a. such level(s) with respect to a reference value determined in a urine sample from the same subject at an earlier time point are indicative that the tuberculosis condition/disease is progressing; and/or
b. such level(s) with respect to a reference value determined in a urine sample from the same subject at an earlier time point are indicative that the respiratory infections caused by S. pneumoniae condition/disease is progressing.

15. The method of claim 14, wherein the biomarkers identified are the levels of at least Citrate, creatinine, mannitol, hippurate, glucose, Phenylalanine, Creatine and 2-Aminoadipic Acid.

16. A computer program for assigning a patient into a specific group of patients according to any of the methods of claims 1 to 7, after introducing the levels of the biomarkers identified in any of claims 1 to 7 into said program.

17. A device comprising the computer program of claim 16, and the use of said device for the diagnosis of tuberculosis in a human subject.
